(19) United States
(12) Patent Application Publication
(10) Pub. No.: US 2022/0001119 A1
(43) Pub. Date: Jan. 6, 2022

(54) APPARATUS AND METHODS FOR MULTI-CHAMBER, MULTI-ATOMIZER VAPORIZATION DEVICES

(71) Applicant: HEXO OPERATIONS INC., Ottawa (CA)

(72) Inventors: Patrick WOODS, Ottawa (CA); Max ALSAYAR, Ottawa (CA); Guy DEGRACE, Ottawa (CA)

(21) Appl. No.: 17/293,544

(22) PCT Filed: Nov. 15, 2019
(86) PCT No.: PCT/CA2019/051632

§ 371 Date: May 13, 2021

Related U.S. Application Data
(60) Provisional application No. 62/768,309, filed on Nov. 16, 2018.

(51) Int. Cl. A61M 11/04; A24F 40/30; A24F 40/42; A24F 40/485; A24F 40/60; A61K 31/05

(52) U.S. Cl. CPC A61M 11/04; A24F 40/30; A24F 40/42; A61M 2205/3334; A24F 40/60; A61K 31/05; A24F 40/485

(57) ABSTRACT

A vaporization device includes a channel and multiple chambers in fluid communication with the channel. The chambers include a first chamber to store a first vaporization substance, and a second chamber to store a second vaporization substance. The first vaporization substance includes an active ingredient or substance, and the second vaporization substance includes an antidote for the active ingredient or substance.

### CROSS-REFERENCE TO RELATED APPLICATION

This application is related to, and claims priority to, U.S. Provisional Patent Application No. 62/768,309, entitled “APPARATUS AND METHODS FOR MULTI-CHAMBER, MULTI-ATOMIZER VAPORIZATION DEVICES”, and filed on Nov. 16, 2018, the entire contents of which are incorporated by reference herein.

### FIELD

This application relates generally to vaporization devices, and in particular to multi-chamber vaporization devices with multiple atomizers.

### BACKGROUND

A vaporization device is used to vaporize substances for inhalation. These substances are referred to herein as vaporization substances, and could include, for example, cannabis products, tobacco products, herbs, and/or flavorants. In some cases, active substances in cannabis, tobacco, or other plants or materials extracted to generate concentrates are used as vaporization substances. These substances could include cannabinoids from cannabis, and nicotine from tobacco. In other cases, synthetic substances are artificially manufactured. Terpenes are common flavorant vaporization substances, and could be generated from natural essential oils or artificially.

Vaporization substances could be in the form of loose leaf in the case of cannabis, tobacco, and herbs, for example, or in the form of concentrates or derivative products such as liquids, waxes, or gels, for example. Vaporization substances, whether intended for flavor or some other effect, could be mixed with other compounds such as propylene glycol, glycerin, medium chain triglyceride (MCT) oil and/or water to adjust the viscosity of a final vaporization substance.

In a vaporization device, the vaporization substance is heated to the vaporization point of one or more constituent ingredients or substances. This produces a vapor, which may also be referred to as an aerosol. The vapor is then inhaled by a user through an air channel that is provided in the vaporization device, and often through a hose or pipe that is part of or attached to the vaporization device.

### SUMMARY

Conventional vaporization devices include a single chamber for storing a vaporization substance. However, vaporization devices with multiple chambers for storing vaporization substances could be desirable. For example, multiple chambers could include different vaporization substances for mixing before vaporization. The vaporization substances in different chambers could be dispensed and vaporized individually, and need not necessarily be mixed. For example, one vaporization substance could include an antidote for another vaporization substance, and the antidote could be vaporized at times when a user wishes to reduce the effects of the other vaporization substance.

According to an aspect of the present disclosure, an apparatus includes a channel and multiple chambers in fluid communication with the channel. The chambers include a first chamber to store a first vaporization substance, and a second chamber to store a second vaporization substance. The first vaporization substance includes an active substance, and the second vaporization substance includes an antidote for the active substance.

The first vaporization substance could include at least one of: a dry substance, a liquid, a gel and a wax, and similarly the second vaporization substance could include at least one of: a dry substance, a liquid, a gel and a wax.

In an embodiment, the active substance is or includes a cannabinoid.

The antidote could be or include, for example, cannabidiol.

At least one of the chambers could include an engagement structure to engage with a complementary engagement structure of the apparatus.

Such an apparatus could include a first atomizer, in fluid communication with the first chamber and the channel, to vaporize the first vaporization substance; and a second atomizer, in fluid communication with the second chamber and the channel, to vaporize the second vaporization substance.

The first atomizer could be inside the first chamber, and/or similarly the second atomizer could be inside the second chamber.

The channel could include multiple channels, with the first atomizer being in fluid communication with a first channel of the multiple channels and the second atomizer being in fluid communication with a second channel of the multiple channels.

The first channel could include a first mouthpiece and the second channel could include a second mouthpiece.

In another embodiment, the first channel and the second channel are in fluid communication with a third channel of the multiple channels, and the third channel includes a mouthpiece.

An apparatus could include a first vaporization substance regulator to control movement of the first vaporization substance from the first chamber, and a second vaporization substance regulator to control movement of the second vaporization substance from the second chamber. In embodiments, at least one of the first vaporization substance regulator and the second vaporization substance regulator includes a wick, a valve, a pump, and/or a mechanical feed structure. An example of a mechanical feed structure is a screw conveyor.

The first vaporization substance regulator and the second vaporization substance regulator could provide dosage control for the apparatus.

In some embodiments, an apparatus includes a first vapor regulator to control a flow of vapor, from vaporization of the first vaporization substance, in the channel; and a second vapor regulator to control a flow of vapor, from vaporization of the second vaporization substance, in the channel.

At least one of the first vapor regulator and the second vapor regulator could be or include a valve or a pump.

The first vapor regulator and the second vapor regulator could provide dosage control for the apparatus.

An apparatus could include a first power controller to control power to the first atomizer and a second power controller to control power to the second atomizer. At least one of the first power controller and the second power controller could be or include an electrical switch.

In some embodiments, the first chamber includes an indicator of the first vaporization substance, and power to the first atomizer is controlled based on the indicator. The second chamber could also or instead include a second indicator of the second vaporization substance, with power to the second atomizer being controlled based on the second indicator.

An apparatus as disclosed herein could include an input device to controllably administer the antidote. The input device is uniquely associated with the second chamber in some embodiments. Examples of an input device include a button, a switch, a slider, a dial, a rotatable structure, a pressure sensor, an accelerometer, and a strain gauge.

Methods are also contemplated. For example, according to another aspect of the present disclosure, a method involves providing a channel and providing multiple chambers to store respective vaporization substances. The chambers include a first chamber to store a first vaporization substance and a second chamber to store a second vaporization substance. The first vaporization substance includes an active substance, and the second vaporization substance includes an antidote for the active substance.

In some embodiments, a method includes arranging the first chamber and the second chamber in fluid communication with the channel.

The first vaporization substance could include at least one of: a dry substance, a liquid, a gel and a wax. The second vaporization substance could also or instead include at least one of: a liquid, a gel and a wax.

In some embodiments, the active substance is or includes a cannabinoid.

The antidote could be or include cannabidiol.

Providing the chambers could involve providing at least one of the first chamber and the second chamber with an engagement structure to engage with a complementary engagement structure of the apparatus.

A method could then further involve arranging the at least one of the first chamber and the second chamber with the engagement structure engaging with the complementary engagement structure of the apparatus.

Other components could also or instead be provided. For example, a method could involve providing a first atomizer to vaporize the first vaporization substance; and providing a second atomizer to vaporize the second vaporization substance.

Atomizers could be installed, for example, by arranging the first atomizer in fluid communication with the first chamber and the channel; and arranging the second atomizer in fluid communication with the second chamber and the channel.

Providing the first atomizer could involve providing the first atomizer inside the first chamber. Similarly, a method could also or instead involve providing the second atomizer inside the second chamber.

In some embodiments, providing the channel involves providing multiple channels, including a first channel for fluid communication with the first atomizer and a second channel for fluid communication with the second atomizer.

Providing multiple channels could involve providing a first mouthpiece including the first channel and providing a second mouthpiece including the second channel. In another embodiment, providing multiple channels involves providing a mouthpiece that includes a third channel for fluid communication with the first channel and the second channel.

A method could involve providing a first vaporization substance regulator to control movement of the first vaporization substance from the first chamber and providing a second vaporization substance regulator to control movement of the second vaporization substance from the second chamber. Installation of the regulators could involve arranging the first vaporization substance regulator in fluid communication with the first chamber and arranging the first vaporization substance regulator in fluid communication with the first chamber.

At least one of the first vaporization substance regulator and the second vaporization substance regulator could include: a wick, a valve, a pump, and a mechanical feed structure.

Vapor regulators could also or instead be provided. For example, a method could include providing a first vapor regulator to control a flow of vapor, from vaporization of the first vaporization substance, in the channel; and providing a second vapor regulator to control a flow of vapor, from vaporization of the second vaporization substance, in the channel.

A method could also involve arranging the first vapor regulator in fluid communication with the channel; and arranging the second vapor regulator in fluid communication with the channel.

At least one of the first vapor regulator and the second vapor regulator could be or include a valve or a pump.

In some embodiments, a method involves providing a first power controller to control power to the first atomizer and providing a second power controller to control power to the second atomizer. A method could also involve coupling the first power controller to the first atomizer and coupling the second power controller to the second atomizer.

A method could also involve providing an input device to controllably administer the antidote.

Another aspect of the present disclosure relates to a method of use of an apparatus as disclosed herein. Such a method could involve initiating vaporization of the first vaporization substance to produce vapor that includes the active substance, inhaling through the channel the vapor that includes the active substance, initiating vaporization of the second vaporization substance to produce vapor that includes the antidote, and inhaling through the channel the vapor that includes the antidote.

According to a further aspect of the present disclosure, a method involves initiating vaporization of a first vaporization substance that includes an active substance to produce vapor that includes the active substance, inhaling the vapor that includes the active substance, initiating vaporization of a second vaporization substance that includes an antidote for the active substance to produce vapor that includes the antidote, and inhaling the vapor that includes the antidote.

In an embodiment, initiating vaporization of the second vaporization substance involves operating an input device to controllably administer the antidote.

Other aspects and features of embodiments of the present disclosure will become apparent to those ordinarily skilled in the art upon review of the following description.

### BRIEF DESCRIPTION OF THE DRAWINGS

For a more complete understanding of the present disclosure, reference is now made to the following description taken in conjunction with the accompanying drawings, in which:

FIG. 1 is a plan view of an example vaporization device;

FIG. 2 is an isometric view of the vaporization device in FIG. 1;

FIG. 3 is an isometric and partially exploded view of an example multi-chamber vaporization device;

FIG. 4 is a cross-sectional view of the example multi-chamber vaporization device of FIG. 3, along line A-A in FIG. 3;

FIG. 5 is a block diagram of an example vaporization device that enables mixing of multiple vaporization substances prior to vaporization;

FIG. 6A is a block diagram of an example stirring element according to an embodiment;

FIG. 6B is a block diagram of an example stirring element according to another embodiment;

FIG. 7A is a cross-sectional view of a passive mixing channel according to an embodiment;

FIG. 7B is a cross-sectional view of a passive mixing channel according to another embodiment;

FIG. 7C is a cross-sectional view of a passive mixing channel according to yet another embodiment;

FIG. 7D is a cross-sectional view of a passive mixing channel according to a further embodiment;

FIG. 8 is an isometric view of a multi-chamber cartridge according to an embodiment;

FIG. 9 is an isometric and partially exploded view of the multi-chamber cartridge of FIG. 8;

FIG. 10 is a plan view of the multi-chamber cartridge of FIG. 8;

FIG. 11 is a top view of the multi-chamber cartridge of FIG. 8;

FIG. 12 is a cross-sectional view of the example multi-chamber cartridge of FIG. 8, along line B-B in FIG. 11;

FIG. 13 is a cross-sectional and partially exploded view of an example of engagement structures in the multi-chamber cartridge of FIG. 8, along a part of line B-B in

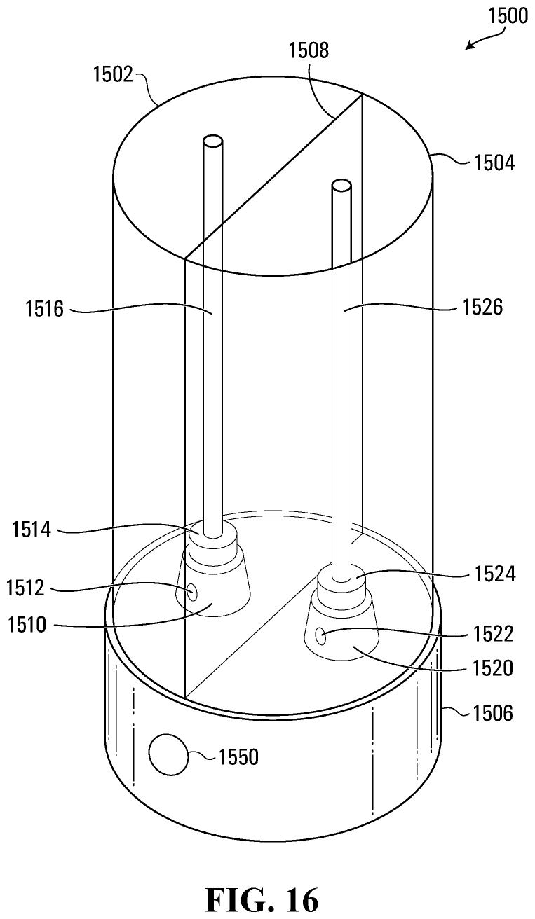

FIG. 11;

FIG. 14 is a block diagram of an example multi-chamber, multi-atomizer vaporization device that enables mixing of vapors produced from multiple vaporization substances;

FIG. 15 is a plan view of a multi-chamber, multi-atomizer cartridge according to an embodiment;

FIG. 16 is a isometric view of the cartridge of FIG. 15;

FIG. 17 is an isometric view of a cap for the cartridge of FIG. 15;

FIG. 18 is a bottom view of the cap of FIG. 17;

FIG. 19 is a cross-sectional view of the cap of FIG. 17, along line C-C in FIG. 18;

FIG. 20 is a flow diagram illustrating a method according to an embodiment;

FIG. 21 is a flow diagram illustrating a method according to another embodiment.

### DETAILED DESCRIPTION

For illustrative purposes, specific example embodiments will be explained in greater detail below in conjunction with the figures. It should be appreciated, however, that the present disclosure provides many applicable concepts that can be embodied in any of a wide variety of specific contexts. The specific embodiments discussed are merely illustrative and do not limit the scope of the present disclosure. For example, embodiments could include additional, different, or fewer features than shown in the drawings. The figures are also not necessarily drawn to scale.

The present disclosure relates, in part, to vaporization devices for vaporization substances that include active ingredients or substances such as one or more cannabinoids or nicotine. However, the vaporization devices described herein could also or instead be used for vaporization substances without an active ingredient or substance. As used herein, the term “cannabinoid” is generally understood to include any chemical compound that acts upon a cannabinoid receptor. Cannabinoids could include endocannabinoids (produced naturally by humans and animals), phytocannabinoids (found in cannabis and some other plants), and synthetic cannabinoids (manufactured artificially).

Examples of phytocannabinoids include, but are not limited to, cannabigerolic acid (CBGA), cannabigerol (CBG), cannabigerol monomethylether (CBGM), cannabigerovarin (CBGV), cannabichromene (CBC), cannabichromevarin (CBCV), cannabidiol (CBD), cannabidiol monomethylether (CBDM), cannabidiol-C4 (CBD-C4), cannabidivarin (CBDV), cannabidiorcol (CBD-C1), delta-9-tetrahydrocannabinol (Δ9-THC), delta-9-tetrahydrocannabinolic acid A (THCA-A), delta-9-tetrahydrocannabionolic acid B (THCA-B), delta-9-tetrahydrocannabinolic acid-C4 (THCA-C4), delta-9-tetrahydrocannabinol-C4, delta-9-tetrahydrocannabivarin (THCV), delta-9-tetrahydrocannabiorcol (THC-C1), delta-7-cis-iso tetrahydrocannabivarin, delta-8-tetrahydrocannabinol (Δ8-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabionol (OTHC), delta-9-cis-tetrahydrocannabinol (cis-THC), 3,4,5,6-tetrahydro-7-hydroxy-alpha-alpha-2-trimethyl-9-n-propyl-2, 6-methano-2H-1-benzoxocin-5-methanol (OH-iso-HHCV), cannabiripsol (CBR), trihydroxy-delta-9-tetrahydrocannabinol (triOH-THC), cannabinol propyl variant (CBNV), and derivatives thereof.

Examples of synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, and quinolinyl esters.

A cannabinoid may be in an acid form or a non-acid form, the latter also being referred to as the decarboxylated form since the non-acid form can be generated by decarboxylating the acid form. Within the context of the present disclosure, where reference is made to a particular cannabinoid, the cannabinoid can be in its acid or non-acid form, or be a mixture of both acid and non-acid forms.

A vaporization substance may comprise a cannabinoid in its pure or isolated form or a source material comprising the cannabinoid. Examples of source materials comprising cannabinoids include, but are not limited to, cannabis or hemp plant material (e.g, flowers, seeds, trichomes, and kief), milled cannabis or hemp plant material, extracts obtained from cannabis or hemp plant material (e.g., resins, waxes and concentrates), and distilled extracts or kief. In some embodiments, pure or isolated cannabinoids and/or source materials comprising cannabinoids may be combined with water, lipids, hydrocarbons (e.g., butane), ethanol, acetone, isopropanol, or mixtures thereof.

In some embodiments, the cannabinoid is tetrahydrocannabinol (THC). THC is only psychoactive in its decarboxylated state. The carboxylic acid form (THCA) is non-psychoactive. Delta-9-tetrahydrocannabinol (Δ9-THC) and delta-8-tetrahydrocannabinol (Δ8-THC) produce the effects associated with cannabis by binding to the CB1 cannabinoid receptors in the brain.

In some embodiments, the cannabinoid is cannabidiol (CBD). The terms “cannabidiol” or “CBD” are generally understood to refer to one or more of the following compounds, and, unless a particular other stereoisomer or stereoisomers are specified, includes the compound “Δ2-cannabidiol.” These compounds are: (1) Δ5-cannabidiol (2-(6-isopropenyl-3-methyl-5-cyclohexen-1-yl)-5-pentyl-1,3-benzenediol); (2) Δ4-cannabidiol (2-(6-isopropenyl-3-methyl-4-cyclohexen-1-yl)-5-pentyl-1,3-benzenediol); (3) Δ3-cannabidiol (2-(6-isopropenyl-3-methyl-3-cyclohexen-1-yl)-5-pentyl-1,3-benzenediol); (4) Δ3,7-cannabidiol (2-(6-isopropenyl-3-methylenecyclohex-1-yl)-5-pentyl-1,3-benzenediol); (5) Δ2-cannabidiol (2-(6-isopropenyl-3-methyl-2-cyclohexen-1-yl)-5-pentyl-1,3-benzenediol); (6) Δ1-cannabidiol (2-(6-isopropenyl-3-methyl-1-cyclohexen-1-yl)-5-pentyl-1,3-benzenediol); and (7) Δ6-cannabidiol (2-(6-isopropenyl-3-methyl-6-cyclohexen-1-yl)-5-pentyl-1,3-benzenediol).

In some embodiments, the cannabinoid is a mixture of tetrahydrocannabinol (THC) and cannabidiol (CBD). The w/w ratio of THC to CBD of a vaporization substance may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1, or about 1000:1.

In some embodiments, a vaporization substance may include products of cannabinoid metabolism, including 11-hydroxy-Δ9-tetrahydrocannabinol (11-OH-THC).

These particulars of cannabinoids are intended solely for illustrative purposes. Other embodiments are also contemplated.

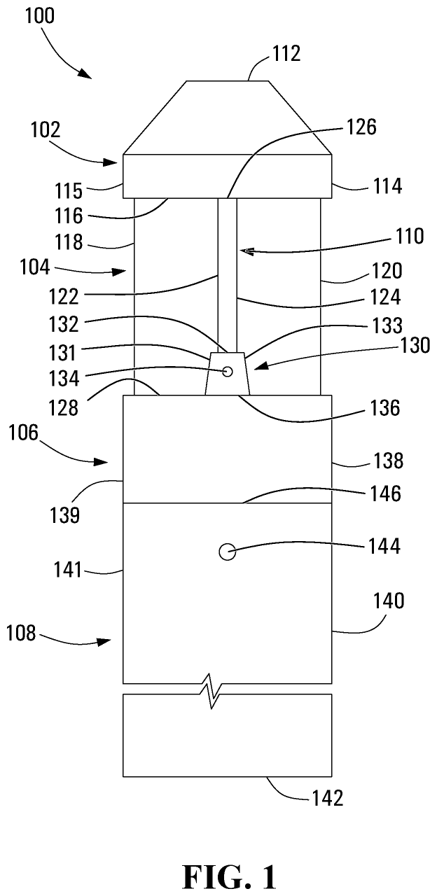

FIG. 1 is a plan view of an example vaporization device 100. In FIG. 1, the vaporization device 100 is viewed from the side. The vaporization device 100 could also be referred to as a vaporizer, a vaporizer pen, a vape pen or an electronic or “e-” cigarette, for example. The vaporizer 100 includes a cap 102, a chamber 104, a base 106 and a battery compartment 108.

The cap 102 is an example of a lid or cover, and includes a tip 112 and sidewalls 114 and 115, which could be sides or parts of the same cylindrical sidewall in some embodiments. The cap 102, in addition to sealing an end of an interior space of the chamber 104, could also provide a mouthpiece through which a user can draw vapor from the vaporization device 100. The mouthpiece could be tapered, as shown, or otherwise shaped for a user's comfort. The present disclosure is not limited to any particular shape of the cap 102.

The cap 102 could be made from one or more materials including metals, plastics, elastomers and ceramics, for example. However, other materials could also or instead be used.

In other embodiments, the mouthpiece could be separate from the cap. For example, the cap could be connected to the mouthpiece by a hose or pipe. The hose or pipe could accommodate the flow of vapor from the cap to the mouthpiece. The hose or pipe could also be flexible, allowing a user to orient the mouthpiece independently from the cap.

The chamber 104 is an example of a vessel to store a vaporization substance prior to vaporization. Although embodiments are described herein primarily in the context of vaporization liquids such as oil concentrates, in general a chamber may store other forms of vaporization substances, including waxes and gels, for example. Vaporization substances with water-based carriers are also contemplated. A vaporization device could be capable of vaporizing water-based carriers with emulsified cannabinoids, for example. In some embodiments, chambers could contain dry vaporization substances. The chamber 104 could also be referred to as a container, a housing or a tank.

The chamber 104 includes outer walls 118 and 120. The outer walls 118 and 120 of the chamber 104 could be made from one or more transparent or translucent materials, such as tempered glass or plastics, in order to enable a user to visibly determine the quantity of vaporization substance in the chamber. The outer walls 118 and 120 could instead be made from one or more opaque materials such as metal alloys, plastics or ceramics, to protect the vaporization substance from degradation by ultraviolet radiation, for example. The outer walls 118 and 120 of the chamber 104 could include markings to aid the user in determining the quantity of vaporization liquid in the chamber. The chamber 104 could be any of a number of different heights. Although multiple outer walls are shown in FIG. 1 at 118 and 120, the chamber 104 is perhaps most often cylindrical, with a single outer wall.

The chamber 104 engages the cap 102, and could be coupled to the cap, via an engagement or connection at 116. A gasket or other sealing member could be provided between the chamber 104 and the cap 102 to seal the vaporization substance in the chamber.

Some chambers are “non-recloseable” or “disposable” and cannot be opened after initial filling. Such chambers are permanently sealed once closed. Others are recloseable chambers in which the engagement at 116, between the cap 102 and the chamber 104, is releasable. For example, the cap 102 could be a cover that releasably engages the chamber 104 and seals a vaporization substance in the chamber 104. A releasable engagement could include, for example, a threaded engagement or other type of connection, or an abutment between the chamber 104 and the cap 102, without necessarily an actual connection between the chamber and the cap. Such a releasable engagement permits the cap 102 to be disengaged or removed from the chamber 104 so that the chamber can be cleaned, emptied, and/or filled with a vaporization substance, for example. The cap 102 could then re-engage with the chamber 104 to seal the vaporization substance inside the chamber.

FIG. 1 also illustrates a stem 110 inside the chamber 104. The stem 110 is a hollow tube or channel through which vapor can be drawn into and through cap 102. The stem 110 may also be referred to as a central column, a central post, a chimney, a hose or a pipe. The stem 110 includes outer walls 122 and 124, although in many embodiments the stem will likely be cylindrical, with a single outer wall. Materials such as stainless steel, other metal alloys, plastics and ceramics could be used for stems such as the stem 110. The stem 110 couples the cap 102 via an engagement or connection 126. Similar to the engagement or connection 116, the engagement or connection 126 could be a releasable engagement or connection that includes a releasable engagement between the stem 110 and the cap 102. In some embodiments, the engagement 126 is in the form of, or includes, a releasable connection.

Although labeled separately in FIG. 1, the engagements at 116 and 126 are operationally related in some embodiments. For example, screwing the cap 102 onto the stem 110 could also engage the cap with the chamber 104, or similarly screwing the cap onto the chamber could also engage the cap with the stem.

An atomizer 130 is provided at the base of the stem 110 inside the chamber 104. The atomizer 130 may also be referred to as a heating element, a core, or a ceramic core. The atomizer 130 includes sidewalls 131 and 133, which could actually be a single cylindrical or frustoconical wall in some embodiments, and one or more wicking holes or intake holes, one of which is shown at 134. The sidewalls of the atomizer 130 could be made from a metal alloy such as stainless steel, for example. The sidewalls 131 and 133 of the atomizer 130 could be made from the same material as the stem 110, or from different materials.

The atomizer 130 engages, and could couple with, the stem 110 via an engagement 132, and with the base 106 via an engagement 136. Although the engagements 132 and 136 could be releasable, the stem 110, the atomizer 130, and the base 106 could be permanently attached together. The atomizer sidewalls 131 and 133 could even be formed with the stem 110 as an integrated single physical component.

In general, the atomizer 130 converts the vaporization substance in the chamber 104 into a vapor, which a user draws from the vaporization device 100 through the stem 110 and the cap 102. Vaporization liquid, for example, could be drawn into the atomizer 130 through the wicking hole 134 and a wick. The atomizer 130 could include a heating element, such as a resistance coil around a ceramic wick, to perform the conversion of vaporization liquid into vapor. A ceramic atomizer could have an integrated heating element such as a coiled wire inside the ceramic, similar to rebar in concrete, in addition to or instead of being wrapped in a coiled wire.

In some embodiments, the combination of the atomizer 130 and the chamber 104 is referred to as a cartomizer.

The base 106 supplies power to the atomizer 130, and may also be referred to as an atomizer base. The base 106 includes sidewalls 138 and 139, which could be a single sidewall such as a cylindrical sidewall. The base 106 engages, and could also be coupled to, the chamber 104 via an engagement 128. The engagement 128 could be a fixed connection. However, in some embodiments, the engagement 128 is a releasable engagement, and the base 106 could be considered a form of a cover that releasably engages the chamber 104 and seals a vaporization substance in the chamber 104. In such embodiments, the engagement 128 could include a threaded engagement, a threaded connection, or an abutment between the chamber 104 and the base 106, for example. A gasket or other sealing member could be provided between the chamber 104 and the base 106 to seal the vaporization substance in the chamber. Such a releasable engagement enables removal or disengagement of the base 106 from the chamber 104 to permit access to the interior of the chamber, so that the chamber can be emptied, cleaned, and/or filled with a vaporization substance, for example. The base 106 could then re-engage with the chamber 104 to seal the vaporization substance inside the chamber.

The base 106 generally includes circuitry to supply power to the atomizer 130. For example, the base 106 could include electrical contacts that connect to corresponding electrical contacts in the battery compartment 108. The base 106 could further include electrical contacts that connect to corresponding electrical contacts in the atomizer 130. The base 106 could reduce, regulate or otherwise control the power/voltage/current output from the battery compartment 108. However, this functionality could also or instead be provided by the battery compartment 108 itself. The base 106 could be made from one or more materials including metals, plastics, elastomers and ceramics, for example, to carry or otherwise support other base components such as contacts and/or circuitry. However, other materials could also or instead be used.

The combination of a cap 102, a chamber 104, a stem 110, an atomizer 130, and a base 106 is often referred to as a cartridge or “cart”.

The battery compartment 108 could also be referred to as a battery housing. The battery compartment 108 includes sidewalls 140 and 141, a bottom 142 and a button 144. The sidewalls 140 and 141, as noted above for other sidewalls, could be a single wall such as a cylindrical sidewall. The battery compartment 108 engages, and could also couple to, the base 106 via an engagement 146. The engagement 146 could be a releasable engagement such as a threaded connection or a magnetic connection, to provide access to the inside of the battery compartment 108. The battery compartment 108 could include single-use batteries or rechargeable batteries such as lithium-ion batteries. A releasable engagement 146 enables replacement of single-use batteries and/or removal of rechargeable batteries for charging, for example. In some embodiments, rechargeable batteries could be recharged by an internal battery charger in the battery compartment 108 without removing them from the vaporization device 100. A charging port (not shown) could be provided in the bottom 142 or a sidewall 140, 141, for example. The battery compartment 108 could be made from the same material(s) as the base 106 or from one or more different materials.

The button 144 is one example of a user input device, which could be implemented in any of various ways. Examples include a physical or mechanical button or switch such as a push button. A touch sensitive element such as a capacitive touch sensor could also or instead be used. A user input device need not necessarily require movement of a physical or mechanical element.

Although shown in FIG. 1 as a closed or flush engagement, the engagement 146 between the base 106 and the battery compartment 108 need not necessarily be entirely closed. A gap between outer walls of the base 106 and the battery compartment 108 at the engagement 146, for example, could provide an air intake path to one or more air holes or apertures in the base that are in fluid communication with the interior of the stem 110. An air intake path could also or instead be provided in other ways, such as through one or more apertures in a sidewall 138, 139, elsewhere in the base 106, and/or in the battery compartment 108. When a user draws on a mouthpiece, air could be pulled through the air intake path into the stem 110, to mix with vapor formed by the atomizer 130.

The battery compartment 108 powers the vaporization device 100 and allows powered components of the vaporization device, including at least the atomizer 130, to operate. Other powered components could include, for example, one or more light-emitting diodes (LEDs), speakers and/or other indicators of device power status (on/off), device usage status (on when a user is drawing vapor), etc. In some embodiments, speakers and/or other audible indicators could produce long, short, or intermittent “beep” sounds as a form of indicator of different conditions. Haptic feedback could also or instead be used to provide status or condition indicators. Varying vibrations and/or pulses, for example, could indicate different statuses or actions in a vaporization device, such as on/off, currently vaporizing, power source connected, etc. Haptic feedback could be provided using small electric motors as in devices such as mobile phones, other electrical and/or mechanical means, or even magnetic means such as one or more controlled electronic magnets.

As noted above, in some embodiments, the cap 102, the chamber 104, the stem 110, the atomizer 130, the base 106 and/or the battery compartment 108 are cylindrical in shape or otherwise shaped in a way such that sidewalls that are separately labeled in FIG. 1 could be formed by a single sidewall. In these embodiments, the sidewalls 114 and 115 represent sides of the same sidewall. Similar comments apply to outer walls 118 and 120, sidewalls 131 and 133, outer walls 122 and 124, sidewalls 138 and 139, sidewalls 140 and 141, and other walls that are shown in other drawings and/or described herein. However, in general, caps, chambers, stems, atomizers, bases and/or battery compartments that are not cylindrical in shape are also contemplated. For example, these components could be rectangular, triangular, or otherwise shaped.

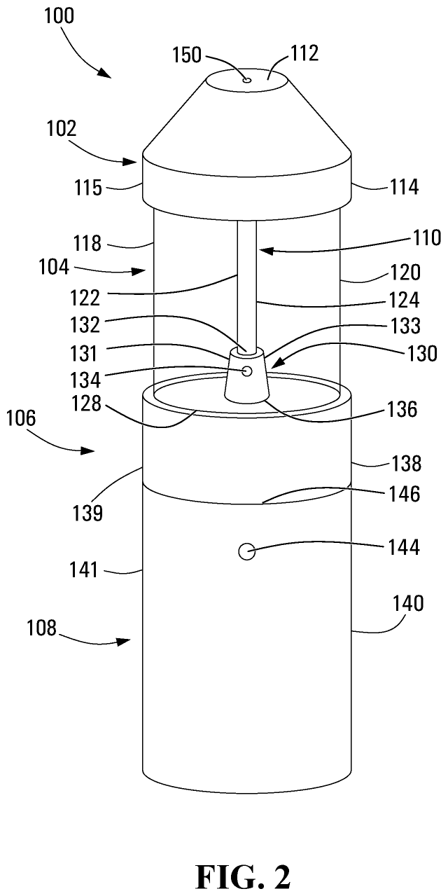

FIG. 2 is an isometric view of the vaporization device 100. In FIG. 2, the cap 102, the chamber 104, the stem 110, the atomizer 130, the base 106, and the battery compartment 108 are illustrated as being cylindrical in shape. However, as noted above, this is not necessarily the case in other vaporization devices. FIG. 2 also illustrates a hole 150 through the tip 112 in the cap 102. The hole 150 could be coupled to the stem 110 through a channel in the cap 102. The hole 150 allows a user to draw vapor through the cap 102. In some embodiments, a user operates the button 144 to vaporize a vaporization substance. Other vaporization devices could be automatically activated to supply power from the battery compartment 108 to powered components of the vaporization device when a user inhales through the hole 150. In such embodiments, a button 144 need not be operated to use a vaporization device, and need not necessarily even be provided.

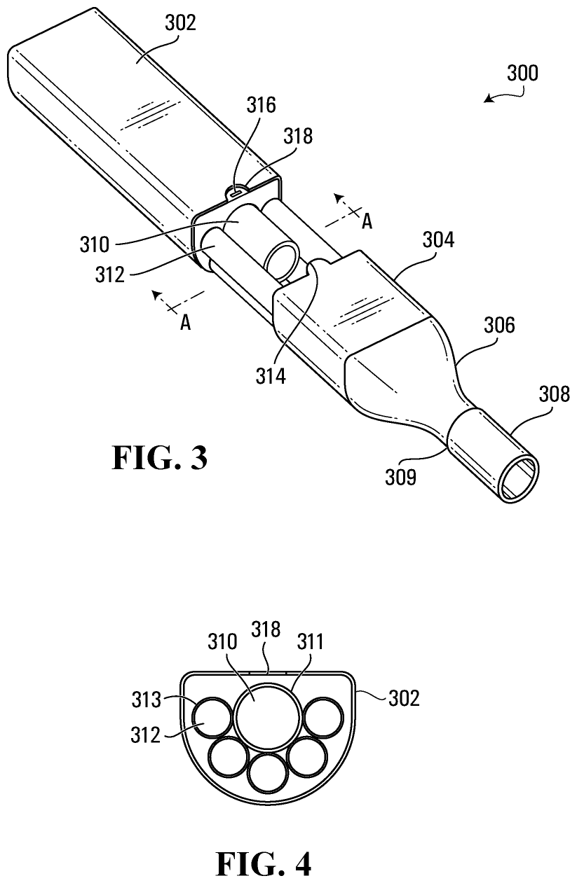

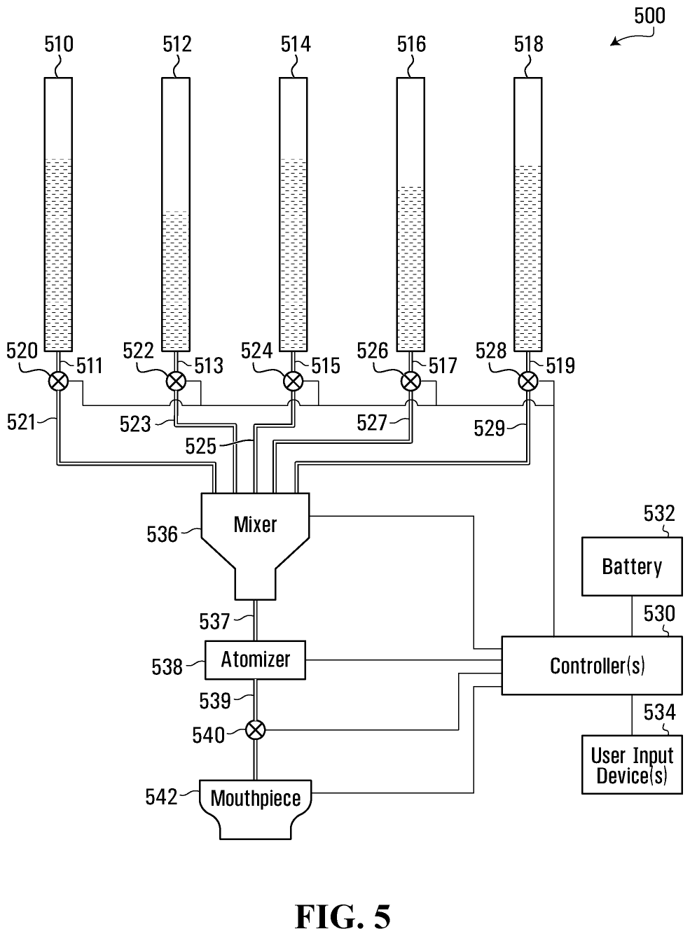

FIG. 3 is an isometric and partially exploded view of an example multi-chamber vaporization device, and FIG. 4 is a cross-sectional view of the example multi-chamber vaporization device along line A-A in FIG. 3. The vaporization device 300 has a multi-part body, with a main body 302 and a removable cover 304. The main body 302 and the cover 304 could be made from the same material(s) or different materials, including one or more of metals, plastics, elastomers and ceramics, for example. However, other materials could also or instead be used.

The main body 302 and the cover 304 include compartments to receive vaporization substance chambers 312 and a channel 310. The compartments in the main body 302 are shown at 311, 313 in FIG. 4, and the cover 304 also includes such compartments. The cover 304 tapers at 306 to a mouthpiece 308 in the example shown, and the mouthpiece is in fluid communication with the channel 310. The main body 302 could at least partially carry or otherwise support components such as the channel 310 and the chambers 312 as shown, and other components such as one or more batteries, electrical contacts, and/or circuitry. Similarly, the cover 304 could at least partially carry or otherwise support components such as the channel 310 and the chambers 312, as well as the mouthpiece 308.

Various channels such as the channel 310 enable fluid flow through a vaporization apparatus such as a vaporization device, or at least parts thereof. Such fluid may include air, at an intake side of an atomizer for example, or a mixture of air and vapor upstream of an atomizer when the atomizer is operating to vaporize a vaporization substance. Fluid flow channels may also be referred to as air channels, but are referenced herein primarily as channels.

The mouthpiece 308 could be made from the same material(s) as the remainder of the cover 304, and could even be integrated with the cover. In the embodiment shown, the mouthpiece 308 engages with the remainder of the cover 304 at an engagement or connection 309. This engagement or connection 309 could be fixed, which might be preferable in embodiments in which the mouthpiece 308 is cylindrical as shown. In other embodiments, a rotatable or otherwise movable engagement or connection 309 might be preferred, so that a user can position the mouthpiece 308 in any preferred orientation relative to the main body 302 and/or the remainder of the cover 304.

Materials such as stainless steel, other metal alloys, plastics and ceramics could be used for the channel 310.

The chambers 312 could be made, at least in part, from one or more materials such as tempered glass, plastics, metal alloys, and/or ceramics. The chambers 312 could be substantially similar to chamber 104 shown by way of example in FIGS. 1 and 2, and could be coupled to other parts that are made from different materials. In some embodiments, the chambers 312 are cartridges that each include a base, an atomizer, a stem, and a cap.

The cover 304 is removable or releasable from the main body 302. In the example shown in FIG. 3, a tab 314 on the cover 304 could be provided with a protrusion on its inner surface, to engage with a groove or slot 316 in the main body 302 when the vaporization device 300 is assembled or closed. This is an example of a releasable engagement between the main body 302 and the cover 304. The cover 304 could be removed, to install or remove chambers 312 and/or for cleaning the device 300 for example, by pulling the cover 304 away from the main body 302 with sufficient force to release the protrusion on the tab 314 from the slot or groove 316. Removal of the cover 304 in the embodiment shown could also or instead involve prying the tab 314 away from the slot or groove 316 to release the tab protrusion and allow the cover to be removed.

The main body 302 could include a structure 318 to accommodate the tab 314, so that the outer surface of the tab is flush with the outer surface of the main body when the device 300 is assembled. The structure 318 could be larger than the tab 314 in some embodiments, to provide clearance for a user to insert a fingernail or tool to pry the tab away from the slot or groove 316 when the cover 304 is to be removed.

In operation, one or more batteries inside the main body 302 provide power to one or more atomizers, which vaporize one or more vaporization substances individually and/or one or more mixtures of vaporization substances from multiple chambers 312. Any of various arrangements or implementations are possible, and examples are disclosed herein.

It should be appreciated, however, that the example device 300 is solely for the purpose of illustration. Other embodiments are also contemplated. For example, the channel 310 need not be a separate component and could be integrated or integral with the main body 302 and/or the cover 304. Each chamber 312 could include a stem as shown in FIGS. 1 and 2, and the cover 304 could then include a manifold to fluidly couple each stem to the mouthpiece 308 without the channel 310. The channel 310 and/or the chambers 312 could be accommodated entirely within the main body 302, in which case the cover 304 need not include compartments to receive part of each chamber. Compartments could be implemented in any of various ways, and not only as the bores shown at 311, 313 in FIG. 4. Multiple engagement structures such as the tab 314 and the slot or groove 316 could be provided. Other types of connection or engagement between a main body and a cover, such as a magnetic connection, are also possible. Different shapes or layouts could be implemented, to have a central channel with compartments or structures to accommodate chambers around the central channel, for example. A multi-chamber vaporization device with a hexagonal cross-sectional shape, for example, could accommodate six cartridges or chambers around a central air channel or mixing channel. At least certain shapes could be suitable for other types of releasable engagement between a main body and a cover, such as a threaded engagement for a cylindrical vaporization device.

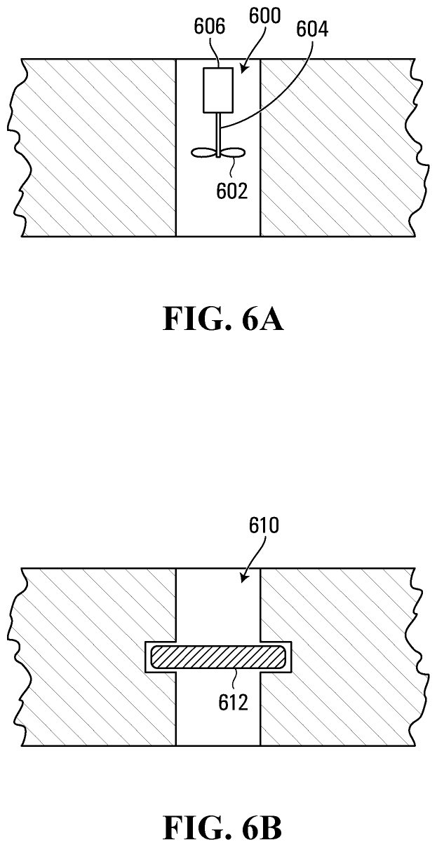

With multiple vaporization substances available in a multi-chamber vaporization device, more than one vaporization substance could be vaporized for inhalation. For example, as disclosed herein, multiple vaporization substances could be mixed to form a vaporization substance mixture, with that mixture then being vaporized by an atomizer. FIG. 5 is a block diagram of an example vaporization device that enables mixing of multiple vaporization substances prior to vaporization.

The example device 500 includes multiple chambers 510, 512, 514, 516, 518 to store respective vaporization substances. Examples of vaporization substances and how vaporization substance chambers could be implemented are disclosed elsewhere herein. Vaporization substances could have any of various effects. For example, some vaporization substances could include one or more active ingredients that have a psychoactive effect, whereas others could include flavorants such as terpenes. Some vaporization substances could include an antidote for an active ingredient or substance in another vaporization sub stance.

Valves 520, 522, 524, 526, 528 in the device 500 are examples of regulators in fluid communication with respective chambers 510, 512, 514, 516, 518, through channels 511, 513, 515, 517, 519, to control movement of the vaporization substances from the respective chambers to a mixer 536. The mixer 536 is in fluid communication with the valves 520, 522, 524, 526, 528 through channels 521, 523, 525, 527, 529 in the embodiment shown. Other forms of regulator include, for example, wicks, pumps, and mechanical feed structures such as screw conveyors. A vaporization device could include regulators of different types. Not all types of regulator are necessarily separately controlled. A wick, for example, draws a vaporization substance from a chamber to an atomizer for vaporization, but the wick itself is not controlled.

Regardless of the type(s) of regulators in a multi-chamber device, the regulators may be useful in providing a measure of dosage control. Different vaporization substances could have different levels of active ingredients, and overall dosage of active ingredients in a mixture of vaporization substances could be controlled by controlling the regulators.

Any or all of the valves 520, 522, 524, 526, 528 in the device 500 could be controlled, for example, by one or more user input devices 534. The user input devices 534 could include switches, sliders, dials, and/or other types of input device that enable a user to control vaporization substance flow from each chamber 510, 512, 514, 516, 518. Other input device examples are disclosed elsewhere herein, with reference to the button 144 in FIGS. 1 and 2, for instance.

A user input device 534 need not necessarily be specific to one chamber 510, 512, 514, 516, 518. A single user input device 534 could be used to control vaporization substance flow from multiple chambers 510, 512, 514, 516, 518. Flow from all chambers could be turned on or off with one user input device 534, for example. A user input device 534 could allow a user to scroll through or otherwise select one of a number of different mixing ratios and control vaporization substance flow from multiple chambers 510, 512, 514, 516, 518 according to the selected mixing ratio. In general, one or more user input devices 534 enable a user to control the flow of vaporization substances from their respective chambers 510, 512, 514, 516, 518 to the mixer 536.

Such control of vaporization substance flow regulators could be indirect as shown in FIG. 5, in the sense that the user input device(s) 534 provide input(s) to a controller 530, and the controller controls the regulators, which are valves 520, 522, 524, 526, 528 in the example device 500. The controller 530 could be implemented, for example, using hardware, firmware, one or more components that execute software stored in one or more non-transitory memory devices (not shown), such as a solid-data memory device or a memory device that uses movable and/or even removable storage media. Microprocessors, Application Specific Integrated Circuits (ASICs), Field Programmable Gate Arrays (FPGAs), and Programmable Logic Devices (PLDs) are examples of processing devices that could be used to execute software.

In the illustrated embodiment, a battery 532 provides power to the controller 530, which may then control power to other components of the example device 500. The valves 520, 522, 524, 526, 528 could be controlled in this type of implementation by controlling power to the valves. For example, each valve 520, 522, 524, 526, 528 could be normally closed when not supplied with power, and opened when powered. In other embodiments, power and control are implemented separately. In any case, a controller-based implementation could be used in conjunction at least with valves 520, 522, 524, 526, 528 that are electrically controllable. Other control mechanisms are also possible.

In another embodiment, the valves 520, 522, 524, 526, 528 could be controlled directly by one or more user input devices 534. A user input device 534 could be mechanically, or otherwise, coupled to a valve 520, 522, 524, 526, 528, for example, to physically move one or more valve components to increase or decrease flow of a vaporization substance to the mixer 536.

The mixer 536 is coupled to receive vaporization substances, through channels 521, 523, 525, 527, 529, and mix the vaporization substances to form a vaporization substance mixture. In some embodiments, the mixer 536 is a driven or “active” mixer to actively mix vaporization substances.

For example, the mixer 536 could include a mixing channel to receive the vaporization substances that are to be mixed. Vaporization substances that are to be mixed could flow into a mixing channel and be mixed as they flow through the mixing channel, and/or be temporarily held in a mixing channel during mixing. In the latter example, the mixing channel could itself be considered a chamber or reservoir. A manifold could couple the chambers 510, 512, 514, 516, 518 to the mixing channel, through the channels 511/521, 513/523, 515/525, 517/527, 519/529 and valves 520, 522, 524, 526, 528 in the example device 500, so that vaporization substances from any of the chambers are available for mixing by the mixer 536. Not all vaporization substances need necessarily be mixed. For example, only a subset of available vaporization substances might be mixed. In some embodiments, a vaporization device could also include one or more cartridges with their own atomizers for vaporization separately from vaporization of a vaporization substance mixture.

A stirring element, positioned within a mixing channel or otherwise positioned to contact and mix vaporization substances that flow through a mixing channel for example, could be useful in improving the mixing of vaporization substances. Vaporization substances in the chambers 510, 512, 514, 516, 518 could include different types of vaporization substances, such as liquid(s), dry substance(s) such as flower(s) or powder(s), wax(es), and/or gel(s). Mixing of different vaporization substances, whether of the same type or different types such as one or more liquids and one or more waxes, could be improved by active mixing using a driven stirring element. For example, certain vaporization substances might have higher viscosity than others, and present a particular challenge for mixing prior to vaporization.

The mixer 536 could include a stirring element that is driven electrically, a magnetic stirring element that is driven magnetically, and/or an acoustic stirring element that is driven acoustically, for example.

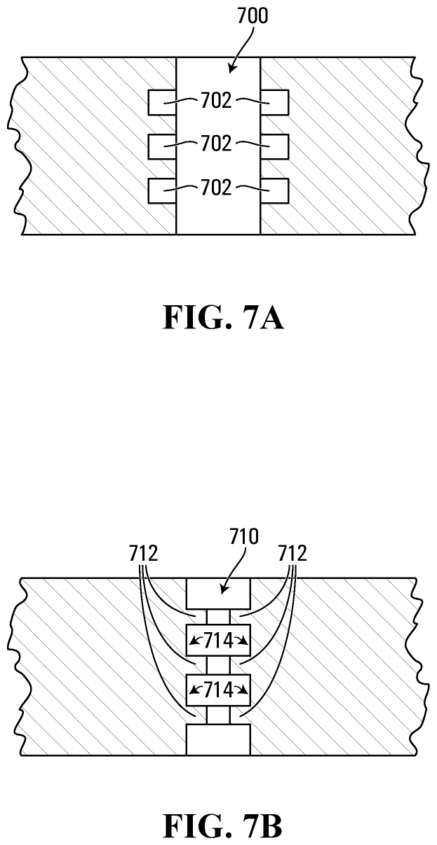

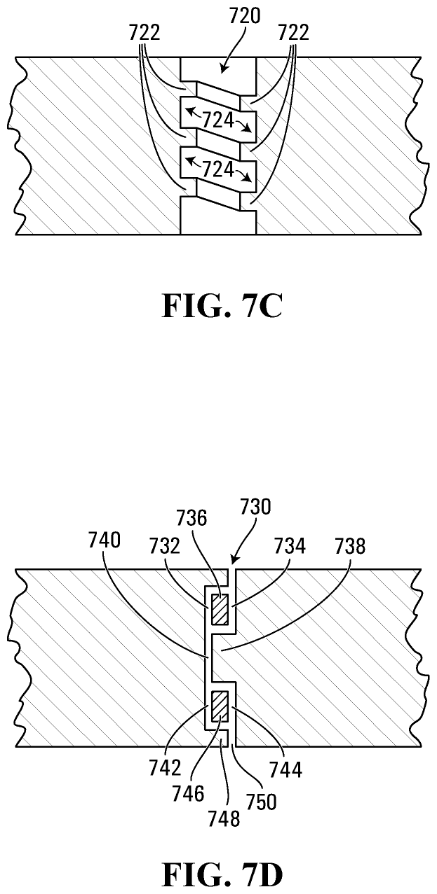

FIG. 6A is a block diagram of an example stirring element according to an embodiment. In FIG. 6A, a stirring element 602 is positioned within a mixing channel 600. Although a flow-through mixing channel 600 open at both ends is shown by way of example, a mixing channel could include a reservoir or other holding structure to temporarily store vaporization substances during mixing and/or after mixing but before vaporization.

The stirring element 602 includes one or more vanes, two in the example shown, coupled to a rotor shaft 604 of an electric motor 606. The electric motor 606 could be supplied with power from one or more batteries of a vaporization device. In some embodiments, a user could operate a switch or other input device to release or actively move, by one or more pumps for example, vaporization substances into the mixing channel 600 and turn on the electric motor 606 to mix the vaporization substances. Release and/or mixing of vaporization substances could instead be initiated when a user draws on a mouthpiece, operates a power button, or otherwise activates the vaporization device for generating vapor for inhalation.

A dual-vane structure as shown in FIG. 6A is intended solely as an illustrative example. Other embodiments could include a stirring element with more than two vanes, a different shape or form of stirring element, and/or multiple stirring elements.

FIG. 6B is a block diagram of an example stirring element according to another embodiment. The embodiment in FIG. 6A is a direct drive embodiment in which the stirring element 602 is directly driven by the electric motor 606. In FIG. 6B, a stirring element 612 is positioned in a mixing channel 610, but is driven indirectly, in particular magnetically or acoustically, rather than through a direct-drive arrangement in which a driving element is in direct physical contact with the stirring element as shown in FIG. 6A.

The stirring element 612 could be a bar, a solid or apertured disc, or an element having another shape, and be driven magnetically or acoustically to rotate, vibrate, reciprocate, or otherwise move within the mixing channel 610.

Acoustic mixing could also or instead be implemented without the use of a mixing or stirring element. For example, an acoustic generator could be coupled to the side walls of the mixing channel 610, causing the side walls to vibrate or otherwise agitate the vaporization substances in the mixing channel.

Other types of active mixing, such as ultrasonic mixing or sonication, could also or instead be used to mix vaporization substances.

The mixer 536 is an active mixer in some embodiments. Examples of active mixers are shown in FIGS. 6A and 6B, and other examples of active mixing and mixers are also disclosed herein. An active mixer, in addition to mixing vaporization substances, could also provide a form of haptic feedback, indicating that the vaporization device is in use and actively mixing vaporization substances. A user holding a vaporization device could feel when an active mixer is operating.

Other embodiments could also or instead involve “passive” mixing of vaporization substances. For passive mixing, the mixer 536 includes multiple mixing elements that are not positively or actively driven, but introduce turbulence into vaporization substance flow or otherwise mix vaporization substances to form a vaporization substance mixture.

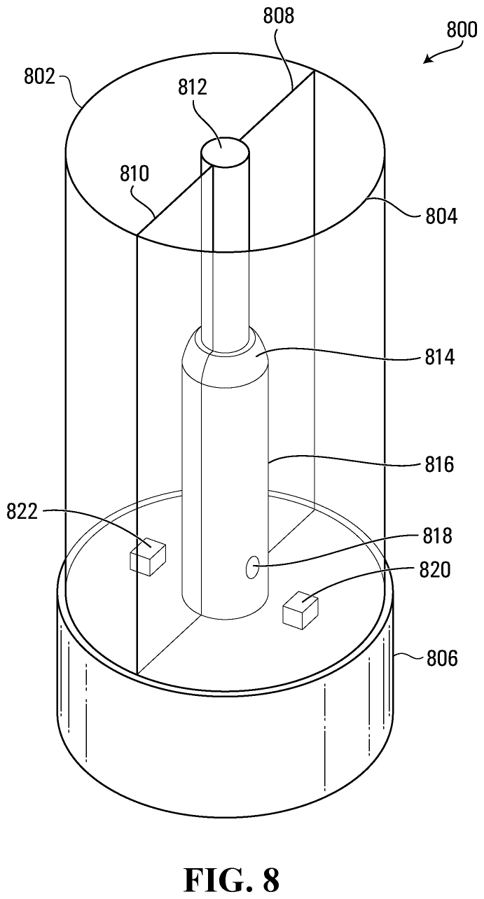

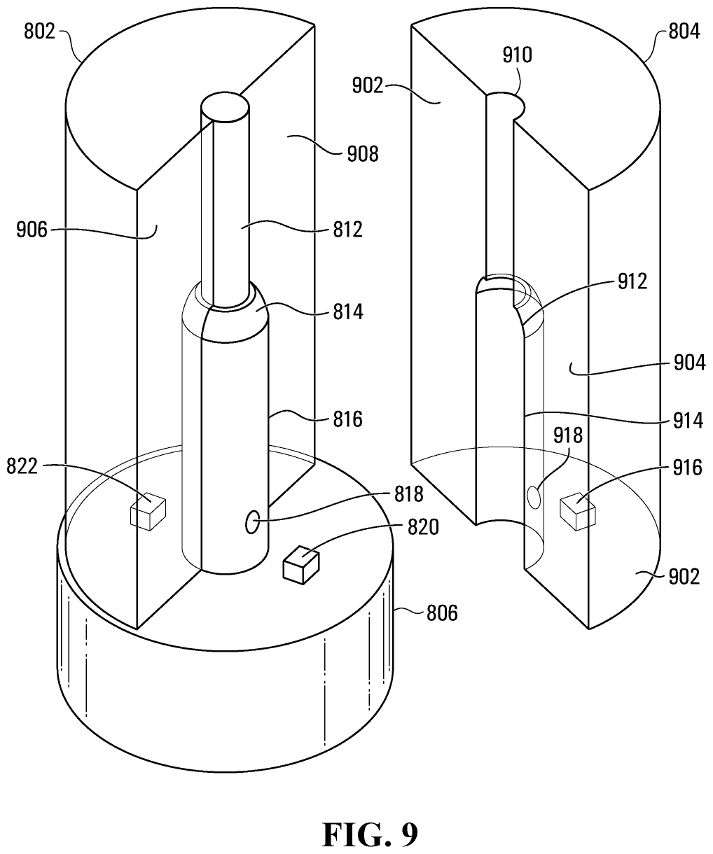

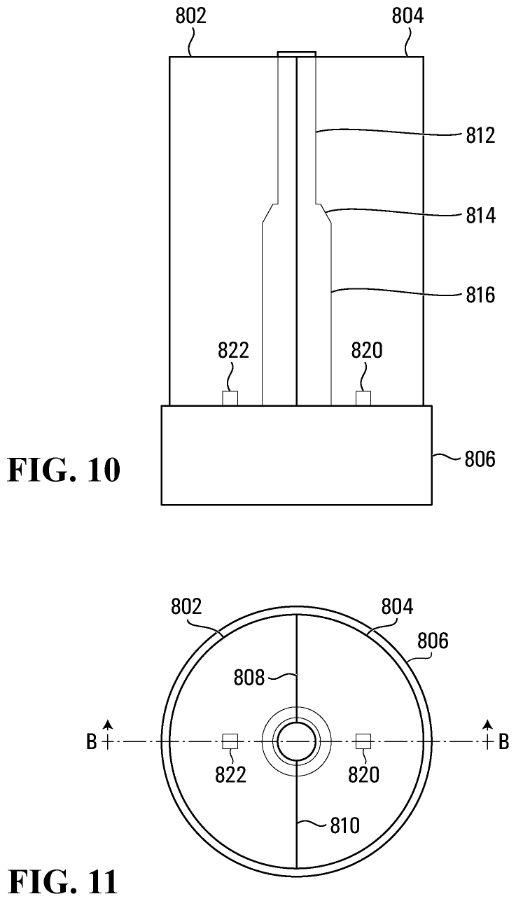

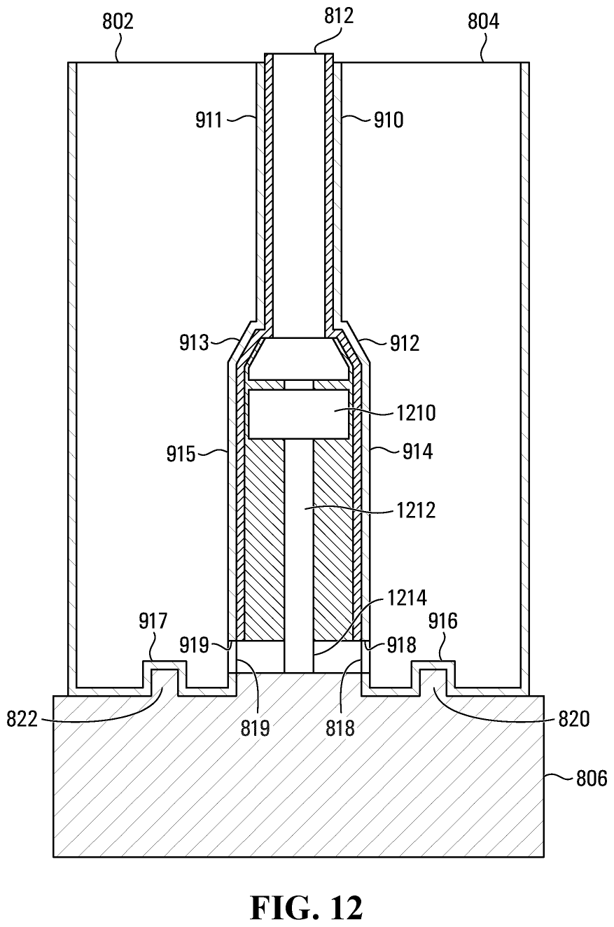

FIG. 7A is a cross-sectional view of a passive mixing channel 700. The mixing channel 700 receives one or more vaporization substances. The vaporization substances, in FIG. 7A and/or in other embodiments, may be pumped, gravity-fed, or otherwise supplied to the mixing channel 700. For example, suction via inhalation could contribute to flow of vaporization substances and/or a vaporization substance mixture, into or through a mixing channel or otherwise to and/or past active or passive mixing elements.

In FIG. 7A, when vaporization substances are to be mixed, multiple vaporization substances are supplied to the mixing channel 700. It should be appreciated, however, that a vaporization device that enables mixing of vaporization substances need not necessarily preclude vaporization of a single vaporization substance. A mixing channel therefore could receive one, or more than one, vaporization substance.

The mixing channel 700 includes multiple mixing elements in the form wells 702. The wells 702, which could be rectangular or cylindrical in shape, for example, are discrete structures formed in the side walls of the mixing channel 700. The wells 702 occupy only a portion of the side walls of the mixing channel 700. Although the wells 702 are illustrated with a fixed shape and spacing in the mixing channel 700, in other embodiments wells could be differently sized and/or spaced, even randomly, in a mixing channel. Moreover, the shape and size of wells in a mixing channel could vary. For example, the side walls of the wells 702 could be tapered or slanted. One or more wells may also or instead extend around an inner surface of a mixing channel, as annular grooves or wells in the case of a cylindrical mixing channel for example.

The wells 702 could increase lateral transport of the vaporizations substances within the mixing channel 700 to aid in mixing. The wells 702 could also or instead produce turbulent flow to aid in mixing. In this sense, the wells 702 passively mix the vaporization substances supplied to the mixing channel 700.

FIG. 7B is a cross-sectional view of a passive mixing channel 710, which receives one or more vaporization substances. The vaporization substances may be pumped, gravity-fed, fed by suction, or otherwise supplied to the mixing channel 710. The mixing channel 710 includes multiple ridges 712 implemented as mixing elements. The ridges 712 could be, for example, annular protrusions around the side wall of the mixing channel 710. The gaps between the ridges 712 could be considered grooves 714. The ridges 712 and/or grooves 714 could take any of a variety of cross-sectional shapes, including rectangular and/or triangular. Although the ridges 712 and grooves 714 are illustrated with a fixed size, spacing and shape in the mixing channel 710, the size, spacing, and/or shape of ridges and/or grooves could vary in other embodiments.

Similar to the wells 702, the ridges 712 and grooves 714 may aid in mixing by increasing lateral transport and/or turbulence of the vaporization substances in the mixing channel 710.

The ridges 712 and grooves 714 are examples of linear mixing elements, in that they extend linearly along the mixing channel 710, in an axial direction in the case of a cylindrical mixing channel. Helical mixing elements are also contemplated. FIG. 7C is a cross-sectional view of a passive mixing channel 720, which includes a helical ridge 722, and a helical groove 724 in the space between turns of the helical ridge. The helical ridge 722 forms a continuous spiral within the mixing channel 720 in the example shown. A helical ridge need not necessarily be continuous, and could include multiple discrete ridge segments. Similarly, the helical groove 724 is continuous in the example shown, but need not necessarily be continuous. The helical ridge 722 and/or helical groove 724 could cause the vaporization substances within the mixing channel 720 to rotate and/or move laterally while they flow through the mixing channel, aiding in mixing.

FIG. 7D is cross-sectional view of another example passive mixing channel 730, which receives one or more vaporization substances. The vaporization substances may be pumped, gravity-fed, fed by suction, or otherwise supplied to the mixing channel 730. The mixing channel 730 includes multiple channels 732, 734, 742 and 744. The mixing channel 730 also includes mixing elements 736, 738, 746 and 748. A stream of vaporization substances received by the mixing channel 730 is split into multiple streams (two in this example) by the mixing element 736. These multiple streams flow through the channels 732 and 734. The mixing element 738 then combines the multiple streams at the end of the channels 732 and 734, into a single stream that flows into and through the channel 740. This process of splitting and combining is repeated once more in the mixing channel 730 using the mixing elements 746 and 748 and the channels 742, 744 and 750. In this sense, the mixing elements 736 and 746, or the channels 732/734 and 742/744, could be considered splitters coupled to combiners in the form of the mixing elements 738 and 748, or the channels 740 and 750. The process of splitting and combining using the mixing elements 736, 738, 746 and 748 could aid in mixing of the vaporization substances. Additional splitters and/or combiners could also be implemented in mixing channel 730 to further mix the vaporization substances. Mixing with a single splitting/combining stage or structure is also possible.

In general, any combination of wells, ridges, grooves, splitters and combiners could be implemented in one or more mixing channels. For example, wells could be added to the channels 732, 734, 740, 742 and/or 744 of the mixing channel 730 to potentially further aid in mixing.

The mixer 536, whether active, passive, or both, is intended to improve mixing between vaporization substances and increase homogeneity of a vaporization substance mixture. Mixture homogeneity could impact the vaporization temperature of a mixture and/or content of a vapor that is generated by vaporizing a mixture, for example, and could therefore be an important parameter when multiple vaporization substances are to be vaporized.

Referring back to FIG. 5, the example vaporization device 500 also includes an atomizer 538, in fluid communication with the mixer 536 through a channel 537, to vaporize the vaporization substance mixture that is formed by the mixer. A power controller to control power to the atomizer 538 could be implemented at 530 in a controller that also provides other control features, and/or in a separate power controller. The power controller could provide on-off power control based on operation of a power button or switch at 534 or a user inhaling on the device 500 through the mouthpiece 542, for example. In some embodiments, different voltages and/or currents could be supplied to the atomizer 538 to enable the atomizer to provide different vaporization temperatures. This type of power control, which could be considered a form of vaporization temperature control, could be provided through one or more user input devices at 534, and/or based on sensing the types of cartridges 510, 512, 514, 516, 518 currently installed in the device 500. In general, the voltage, current, and/or power supplied to the atomizer 538 could be adjusted based on the vaporization substance(s) to be vaporized. The voltage, current, and/or power supplied to the atomizer 538 could also or instead be adjusted based on a desired flow or quantity of vapor produced by the atomizer, which could be selected or otherwise controlled using one or more user input devices 534, for example.

A channel in fluid communication with the atomizer 538 is shown at 539, and a mouthpiece 542 is in fluid communication with the channel so that a user can inhale vapor from the atomizer. In some embodiments, a valve 540 is controllable to regulate or otherwise control the flow of vapor to the mouthpiece 542. The controller 530 could adjust the valve 540 to provide a form of dosage control, for example.

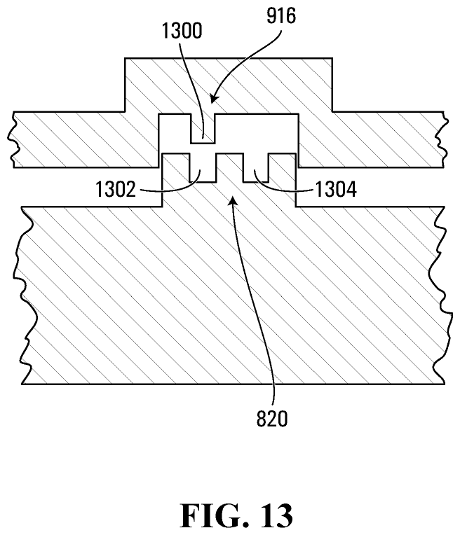

FIG. 5 is a block diagram of an example vaporization device, and FIGS. 6A to 7D show examples of mixers. An example multi-chamber cartridge, which could be used in an embodiment to implement multiple chambers in a vaporization device of the type shown in FIG. 5, is shown in FIGS. 8 to 12.

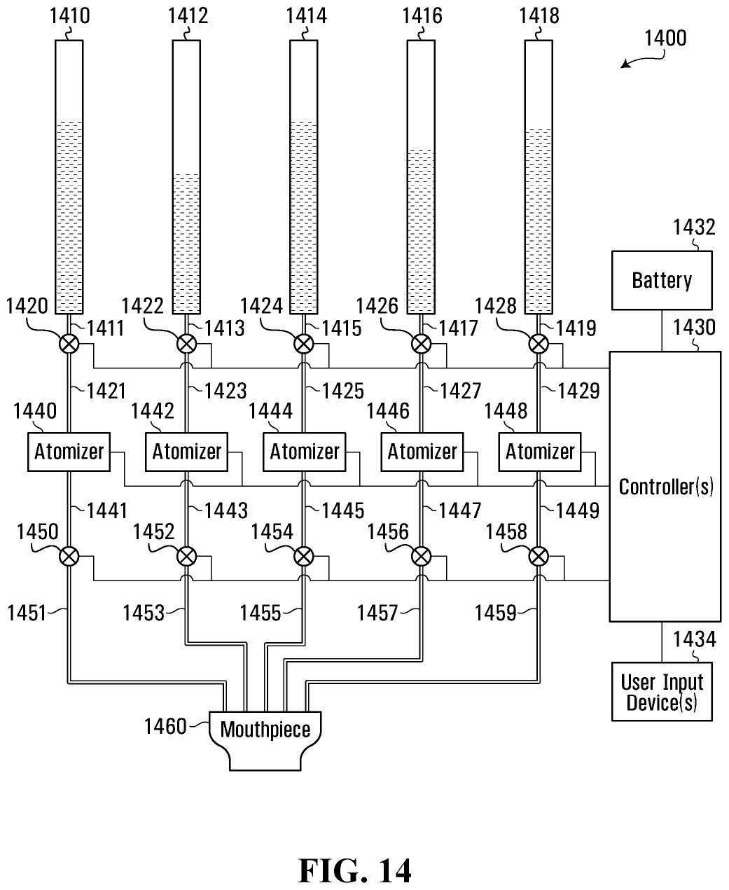

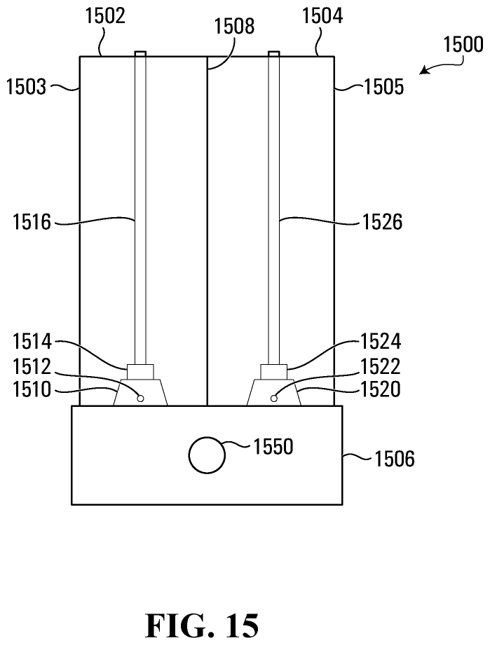

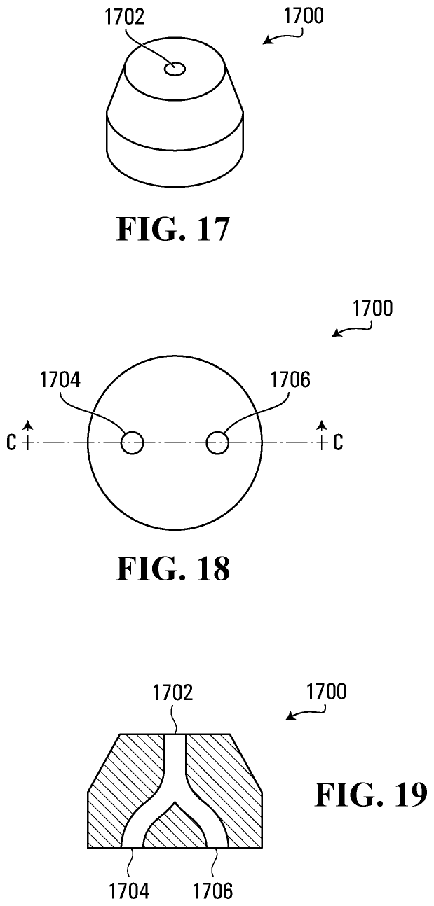

FIG. 8 is an isometric view of a multi-chamber cartridge, FIG. 9 is an isometric and partially exploded view of the multi-chamber cartridge of FIG. 8, FIG. 10 is a plan view of the multi-chamber cartridge of FIG. 8, FIG. 11 is a top view of the multi-chamber cartridge of FIG. 8, and FIG. 12 is a cross-sectional view of the example multi-chamber cartridge of FIG. 8, along line B-B in FIG. 11. Various features referenced in the description below are shown in one or more of these drawings.

The example multi-chamber cartridge 800 includes two chambers 802, 804. Two chambers of equal size are shown by way of example. There could be more than two chambers. Chambers could all be of the same size, or one or more chambers could have a different size from one or more other chambers. The chambers 802, 804 are positioned on a base 806 and could be held in place by friction fit and/or some other type of releasable engagement. A cap (not shown) screwed onto threads at an upper end of a stem 812 could both seal the chambers 802, 804 and also hold them in place on the base 806, for example. Other engagements between a cap and the chambers 802, 804 are possible, and further examples of cap/chamber engagements are provided elsewhere herein.

Examples of materials from which each chamber 802, 804 could be made are provided elsewhere herein. The chambers 802, 804 could include non-recloseable chambers, recloseable chambers, or both a non-recloseable chamber and a recloseable chamber. More generally, a multi-chamber cartridge or multi-chamber vaporization device could include one or more non-recloseable chambers and/or one or more recloseable chambers.

A stem 812 and an atomizer 814 could be implemented as described elsewhere herein, with reference to FIGS. 1 and 2, for example. In FIG. 8, a mixing channel 816 is also provided, and could be implemented by extending atomizer sidewalls relative to the embodiment shown in FIGS. 1 and 2 and providing intake holes or passages 818, 819 at a distal end of the atomizer sidewalls, away from an atomizer end of the mixing channel 816. This is perhaps best shown in FIG. 12, in which the internal position of the atomizer 1210 toward the top of the mixing channel 816 is shown. Parameters such as shape of a mixing channel, any of various dimensions of a mixing channel, distance of an atomizer from a mixing element or mixing channel, and/or distance of a mixing element or mixing channel from vaporization substance intake(s) could be different for different types of vaporization substance, for example. Preferred intake to mixer distance and/or mixer to atomizer distance could be shorter for higher viscosity oils or waxes than for lower viscosity vaporization substances. Mixer to atomizer distance could also or instead take into account expected viscosity of a resultant vaporization substance mixture. More generally, any of various parameters, including not only physical parameters but also or instead other parameters such as mixing element type and/or speed, could be determined or selected based on characteristics of the vaporization substances that are to be mixed and/or expected characteristics of the resultant vaporization substance mixture.

As also shown in FIG. 12, mixing could take place within a passage 1212 inside the mixing channel 816. A part 1214 of the passage 1212 could be perforated or otherwise include intake holes for receiving vaporization substances for mixing. In other embodiments, there is no separate internal passage 1212 inside the mixing channel 816. Active and/or passive mixing could be provided in the mixing channel 816, or otherwise implemented with or without a mixing channel. Any of the example mixers disclosed elsewhere herein could be used to implement vaporization substance mixing in a multi-chamber cartridge.

The atomizer 814 engages, and could couple with, the stem 812 and the mixing channel 816 via respective engagements, and similarly the mixing channel 816 could engage, and could couple with, the base 806 via another engagement. Although any or all of these engagements could be releasable, the stem 812, the atomizer 814, the mixing channel 816, and the base 806 could be permanently attached together. The sidewalls of the atomizer 814 and the mixing channel 816 could even be formed with the stem 812 as an integrated single physical component.

The atomizer 814 converts a mixture of the vaporization substances in the chambers 802, 804 into a vapor, which a user draws through the stem 812. Vaporization substances could be drawn into or otherwise provided to the atomizer 814 through the intake holes 818, 819, and corresponding intake holes 918, 919 in the chambers 802, 804. One or more regulators could also be provided to regulate flows of one or more of the vaporization substances to the mixing channel 816 and/or to the atomizer 814.

The base 806 supplies power to the atomizer 814, and could also supply power to other components such as an active mixer. The base 806 could be implemented, for example, in a similar manner to the base 106 (FIGS. 1 and 2) as described elsewhere herein. The base 806 engages, and could also be coupled to, the chambers 802, 804 via an engagement. The engagement could be a fixed connection or a releasable engagement. In some embodiments, the base 806 could be a form of a cover that releasably engages the chambers 802, 804 and seals one or both of the chambers 802, 804. As shown in FIG. 12, the chambers 802, 804 have respective bottom walls, but in other embodiments the base 806 seals the base end of one or more chambers.

The bottom wall of each chamber 802, 804 includes an engagement structure 916, 917 to engage with a complementary engagement structure 820, 822 on the base 806. In the example shown, the base 806 includes an engagement structure 820, 822 at each chamber position, and therefore only chambers 802, 804 with a complementary engagement structure 916, 917 to accommodate the base engagement structure can be used with the base 806. In other embodiments, only some but not all chambers and chamber positions on a base include an engagement structure. Other shapes, sizes, types, and locations of engagement structures are also contemplated. One or more engagement structures could also or instead be provided on one or more of the mixing channel 816, the atomizer 814, the stem 812, and/or a cap (not shown), for example.

Engagement structures could be useful, for example, for restricting a cartridge or vaporization device to a particular model or type of chamber. Engagement structures could also or instead be useful as an assembly aid, to ensure that chambers are assembled with chamber intake holes 918, 919 aligned with mixing channel intake holes 818, 819, for example.

Multiple chambers could be separated or partitioned by one or more partition walls. With reference to FIG. 8, a central partition between the chambers 802, 804 could be provided in part by partition wall sections 808, 810, in combination with the stem 812, the atomizer 814, and the mixing channel 816. A gasket or other sealing member could be provided between each partition wall section 808, 810 and the stem 812, the atomizer 814, the mixing channel 816, a cylindrical outer chamber wall in the example shown, and either a bottom wall of the chamber or a top surface of the base 806.

Partition wall sections such as 808, 810 could even be movable in some embodiments, to provide for adaptable partitioning of the interior space of a single cartridge chamber into multiple chambers. One or more sealing members could be attached to or otherwise carried by the partition wall sections to provide a seal between adjacent chambers at any position of the partition wall. Grooves, channels, and/or other structures could be provided in a cylindrical outer chamber wall and/or in one or more of the stem 812, the atomizer 814, the mixing channel 816, and a chamber bottom wall or top surface of the base 806 as guides to placement of partition walls at certain positions.

Chambers could also or instead be “self-contained”, as perhaps shown most clearly in FIGS. 9 and 12. A self-contained chamber could include one or more exterior walls, and in particular a curved exterior wall in the example shown, and one or more interior partition walls. In FIGS. 9 and 12, the partition walls have wall sections 902/904, 906/908, each of which abuts another partition wall section when the chambers 802, 804 are assembled together in a vaporization device or cartridge. The partition walls also have sections 910/911, 912/913, 914/915 to accommodate the stem 812, the atomizer 814, and the mixing channel 816, respectively. With self-contained chambers, a sealing member could be provided to seal the intake hole 918, 919 against leakage before a chamber is assembled in a vaporization device or cartridge. The mixing channel 816 could include a structure around a periphery or otherwise in the area of each of the intake holes 818, 819 to rupture or otherwise open a chamber intake hole seal when the chamber is installed in a cartridge, for example. A regulator that controls vaporization substance flow from a chamber for mixing could also or instead be used to reduce or avoid pre-assembly leakage from a chamber. A chamber intake hole seal could extend beyond a periphery of the chamber intake hole to provide a seal against leakage of a vaporization substance from an engagement between a chamber and the mixing channel 816. A separate gasket or other sealing member could be provided for this purpose, on the mixing chamber 816 or on a chamber 802, 804, for example.

FIGS. 8 to 12 represent one example embodiment. Other embodiments are contemplated. For example, a cartridge with a uniform shape from the base to the top of each chamber could be preferred in order to simplify chamber construction or manufacturing. A size transition as shown in FIGS. 8 to 12 between the mixing chamber 816 and the stem 812 could be avoided entirely or relocated into a cover or mouthpiece (not shown). A size transition could instead be made with a frustoconical interior wall, which could at least avoid sharper transitions between partition wall segments or parts. It should also be appreciated that a chamber need not necessarily conform tightly to other components. Tight conformance between components may be preferred to make efficient use of limited physical space, but in other embodiments multiple cylindrical chambers could be assembled to the same base, for example.

A central mixing channel 816 disposed between the chambers 802, 804 is shown in FIGS. 8 to 12. In other embodiments, mixing could also or instead be performed in a mixing channel or reservoir in a base. For example, a base could have one or more input channels in fluid communication with multiple chambers, to feed vaporization substances into a base mixing channel, which is in fluid communication with a single atomizer. The atomizer could also be located in the base and in fluid communication with a stem.

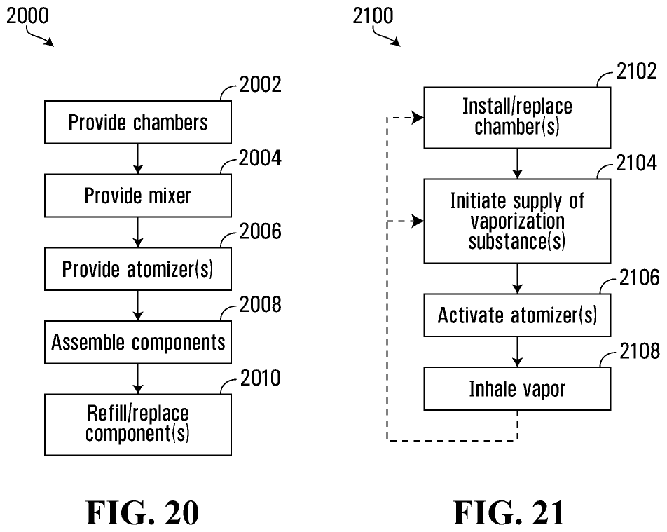

Any of various types of engagement structures could be provided on or in a vaporization device. FIG. 13 is a cross-sectional and partially exploded view of an example of engagement structures in the multi-chamber cartridge of FIG. 8, along a part of line B-B in FIG. 11. In the embodiment illustrated in FIG. 13, the engagement structure 820 includes notches 1302 and 1304, and the complementary engagement structure 916 includes a protrusion 1300. Chambers that include a protrusion 1300, a protrusion (not shown) to engage the notch 1304, both of these protrusions, or no protrusion, could be used with the example engagement structure 820.

Engagement structures that are similar to or different from the examples shown in FIG. 13 could be more specific to particular types of chambers. One or more engagement structures on an apparatus such as a vaporization device could mechanically restrict chambers, cartridges, and/or other components to only specific types. An engagement structure could include one or more features, such as one or more protrusions and/or one or more grooves, with size(s), shape(s), and/or positions to mate only with a particular type of cooperating component with one or more complementary features. A specific threading and pin setup, so that only a specific cartridge type would fit only in an intended device, is another engagement structure example. One or more pins of a particular shape, such as hexagonal, represent a further example of engagement structures to provide cartridge/device specificity. This type of physical or mechanical specificity could be used, for example, to restrict a vaporization device to use with only certain types of chambers or cartridges, which could provide a measure of control over the particular vaporization substances that are available for vaporization by a vaporization device. Certain chambers or cartridges could be restricted to certain positions, which could have regulators, power supply terminals, and/or other features that are specially adapted for those chambers or cartridges, for example.

Engagement structures need not have only a physical function such as controlling correct placement or alignment of a chamber and/or other component or limiting chambers and/or other components to particular types. Engagement structures on different chambers could have different sizes and/or patterns of conductive pins, for example, to enable a vaporization device to detect the type(s) of chambers that have been installed. With reference again to FIG. 13, the protrusion 1300 could include a conductive pin and the notches 1302 and 1304 could include contacts, for example, to provide for detection of an installed chamber or cartridge and/or an installed chamber or cartridge type. Other embodiments are also contemplated, and the notches 1302 and 1304 could include pressure sensors or another type of sensor to detect the presence of a protrusion 1300.

In the example of FIG. 13, the presence of the protrusion 1300 aligned with the notch 1302 and the lack of a protrusion aligned with the notch 1304 could provide information regarding an installed chamber. This information could include the type of vaporization substance stored by the chamber, which could be used by a controller, in a base of a multi-chamber cartridge or elsewhere in a multi-chamber device, for example, to control the voltage, current, and/or power supplied to an atomizer. One or more regulators within a multi-chamber cartridge or device could also or instead be controlled based on the type of vaporization substance stored by the chamber.

Each different type of chamber that is compatible with a multi-chamber cartridge or device could have a unique engagement structure. The two notches 1302 and 1304 in FIG. 13 can detect a maximum of four different types of chambers, including chambers with no protrusions, chambers with two protrusions, chambers with only one protrusion 1300 as shown, and chambers with only one protrusion that corresponds to notch 1304. However, engagement structures with more or fewer notches could be used to enable detection of different numbers of chamber types.

The protrusions and notches illustrated in FIG. 13 are provided by way of example only. Other arrangements, sizes, and shapes of engagement structures that might or might not include protrusions and/or grooves are also contemplated. Although described above primarily in the context of chambers, engagement structures could also or instead be used in conjunction with cartridges and/or other components. Engagement structures are also not in any way limited to localized structures at certain locations on or in an apparatus or component. Different types of chamber or cartridge could have different shapes that will only fit into compartments, such as those shown at 313 in FIG. 4, for example, that have a complementary shape.

Some embodiments described above relate to mixing of vaporization substances prior to vaporization. Vapors produced from different vaporization substances could also or instead be mixed after vaporization.

FIG. 14 is a block diagram of an example multi-chamber, multi-atomizer vaporization device 1400 that enables mixing of vapors produced from multiple vaporization substances. The example device 1400 includes multiple chambers 1410, 1412, 1414, 1416, 1418 to store respective vaporization substances. The chambers 1410, 1412, 1414, 1416, 1418 are in fluid communication with respective valves 1420, 1422, 1424, 1426, 1428 through multiple channels 1411, 1413, 1415, 1417, 1419. These chambers, valves and channels could be similar to the chambers 510, 512, 514, 516, 518, channels 511, 513, 515, 517, 519 and valves 520, 522, 524, 526, 528 described above with reference to FIG. 5, for example. Implementation examples for these components are also disclosed elsewhere herein.

The valves 1420, 1422, 1424, 1426, 1428 are examples of regulators to control movement of the vaporization substances from the respective chambers. Other forms of regulator include, for example, wicks, pumps, and mechanical feed structures such as screw conveyors. As noted elsewhere herein, a vaporization device could include regulators of different types, and not all types of regulator are necessarily controlled.

Any or all of the valves 1420, 1422, 1424, 1426, 1428 in the device 1400 could be controlled, for example, by one or more user input devices 1434 coupled to a controller 1430. The user input devices 1434 could include switches, sliders, dials, and/or other types of input device that enable a user to control vaporization substance flow from each chamber 1410, 1412, 1414, 1416, 1418. Other input device examples are disclosed elsewhere herein, with reference to the button 144 in FIGS. 1 and 2, for instance.

A single user input device 1434 could be used to control vaporization substance flow from multiple chambers 1410, 1412, 1414, 1416, 1418. Flow from all chambers could be turned on or off with one user control device 1434, for example. A user input device 1434 could allow a user to scroll through or otherwise select one of a number of different dosage or flow rates for vaporization substance flow from multiple chambers 1410, 1412, 1414, 1416, 1418 to achieve a desired combination of vapors, or vapor from only one vaporization substance. In general, in some embodiments one or more user input devices 1434 enable a user to control the flow of vaporization substances from chambers 1410, 1412, 1414, 1416, 1418.

As described above with reference to FIG. 5, for example, control of vaporization substance flow regulators could be indirect, with the user input device(s) 1434 providing input(s) to a controller 1430. Examples of how the controller 1430 could be implemented are provided elsewhere herein, in the description of FIG. 5 above, for example.

In the embodiment illustrated in FIG. 14, a battery 1432 provides power to the controller 1430, which may then control power to other components of the example device 1400. The valves 1420, 1422, 1424, 1426, 1428 could be controlled in this type of implementation by controlling power to the valves, for example. In other embodiments, power and control are implemented separately. Other control mechanisms are also possible.

In another embodiment, one or more of the valves 1420, 1422, 1424, 1426, 1428 could be controlled directly by one or more user input devices 1434. A user input device 1434 could be mechanically, or otherwise, coupled to a valve 1420, 1422, 1424, 1426, 1428, for example, to physically move one or more valve components to increase or decrease flow of a vaporization substance from a chamber 1410, 1412, 1414, 1416, 1418. A particular user input devices 1434 could be dedicated or uniquely assigned to a particular chamber 1410, 1412, 1414, 1416, 1418 that stores an antidote, to provide a “panic button”-type safety feature enabling a user to dispense only an antidote to reduce an effect of an active ingredient or substance that has been previously inhaled by the user, for instance.

The valves 1420, 1422, 1424, 1426, 1428 are in fluid communication with respective atomizers 1440, 1442, 1444, 1446, 1448 through multiple channels 1421, 1423, 1425, 1427, 1429. Although FIG. 14 illustrates the chambers 1410, 1412, 1414, 1416, 1418, the valves 1420, 1422, 1424, 1426, 1428, and the atomizers 1440, 1442, 1444, 1446, 1448 in fluid communication through the channels 1411, 1413, 1415, 1417, 1419 and 1421, 1423, 1425, 1427, 1429, it should be appreciated that this is just an example. The channels 1411, 1413, 1415, 1417, 1419, the channels 1421, 1423, 1425, 1427, 1429, and/or possibly other channels disclosed herein, are fluid paths and need not necessarily be separate physical components. A valve and an atomizer could be provided in a cartridge, at the output (bottom) of a chamber, for example. This could improve vaporization substance flow and/or flow control per cartridge by shortening the flow distance for a vaporization substance from a chamber to its atomizer. A shorter distance could also avoid or reduce potential viscosity issues such as significantly different flow times between different chambers and their atomizers.

The atomizers 1440, 1442, 1444, 1446, 1448 are provided to vaporize vaporization substances from respective chambers 1410, 1412, 1414, 1416, 1418. A power controller to control power to the atomizers 1440, 1442, 1444, 1446, 1448 could be implemented in at 1430 in a controller that also provides other control features, and/or in a separate power controller. The power controller could provide on-off power control based on operation of a power button or switch at 1434 or a user inhaling on the device 1400 through the mouthpiece 1460, for example.

In some embodiments, different voltages and/or currents could be supplied to different atomizers 1440, 1442, 1444, 1446, 1448 to enable each atomizer to provide different vaporization temperatures. This type of power control could be considered a form of vaporization temperature control, and could be provided through one or more user input devices at 1434, and/or based on sensing the types of cartridges 1410, 1412, 1414, 1416, 1418 currently installed in the device 1400. In general, the voltage, current, and/or power supplied to any one or more of the atomizers 1440, 1442, 1444, 1446, 1448 could be adjusted based on the vaporization substance to be vaporized in each atomizer. The voltage, current, and/or power supplied to any one or more of the atomizers 1440, 1442, 1444, 1446, 1448 could also or instead be adjusted based on a desired flow or quantity of vapor produced by that atomizer, which could be selected or otherwise controlled using one or more user input devices 1434, for example.

The flow of vapor from any one or more of the atomizers 1440, 1442, 1444, 1446, 1448 could be controlled by controlling valves 1450, 1452, 1454, 1456, 1458, which are in fluid communication with the atomizers through respective channels 1441, 1443, 1445, 1447, 1449. The valves 1450, 1452, 1454, 1456, 1458 are examples of gas or vapor regulators that are controllable to regulate or otherwise control the flow of vapor to the mouthpiece 1460, and could provide a form of dosage control. Other examples of gas regulators include pumps, such as fans. The valves 1450, 1452, 1454, 1456, 1458 could be controlled using the controller 1430. Control options disclosed elsewhere herein, for the valves 1420, 1422, 1424, 1426, 1428 for example, could also or instead be implemented to control any of the valves 1450, 1452, 1454, 1456, 1458.

A mouthpiece 1460 is in fluid communication with the valves 1450, 1452, 1454, 1456, 1458 through multiple channels 1451, 1453, 1455, 1457, 1459. The mouthpiece 1460 enables a user to inhale vapor produced by one or more of the atomizers 1440, 1442, 1444, 1446, 1448. The mouthpiece 1460 could include a mixing channel that is in fluid communication with the channels 1451, 1453, 1455, 1457, 1459, and one or more output holes. A mixing channel could improve mixing of vapor from any or all of the channels 1451, 1453, 1455, 1457, 1459 for inhalation through the output hole. Examples of mixing channels are provided elsewhere herein. Active and/or passive mixing, using any of various types of mixers such as those disclosed elsewhere herein, could be used to mix vapor that is produced by multiple atomizers 1440, 1442, 1444, 1446, 1448. Active mixers could also provide a form of haptic feedback, indicating that the vaporization device is in use and actively mixing vapor for example. Passive mixing could be more effective for mixing vapor than for mixing higher viscosity vaporization substances, but active mixing could also or instead be provided for mixing vapor. Vapor could be drawn into a mixing channel and/or past active or passive mixing or stirring elements via inhalation by a user on the mouthpiece 1460. Vapor flow could also or instead be assisted by at least certain types of vapor regulators such as fans, which could also provide haptic feedback. A stirring element of an active mixer could also or instead be shaped or otherwise configured to drive or at least contribute to vapor flow. One or more vanes of the stirring element 602 (FIG. 6A) or sidewalls of apertures in a rotating apertured disc at 612 (FIG. 6B), for example, could be shaped and/or oriented to increase vapor flow.

The mouthpiece 1460 could include respective output holes for each of the channels 1451, 1453, 1455, 1457, 1459, such that a user can inhale from each output hole at the same time. In this example, mixing of the vapors from different atomizers 1440, 1442, 1444, 1446, 1448 occurs outside the device 1400. In other embodiments, any or all of the channels 1451, 1453, 1455, 1457, 1459 could be in fluid communication with respective mouthpieces.

The valves 1450, 1452, 1454, 1456, 1458 could be particularly useful in implementations that support vaporization from fewer than all of the atomizers 1440, 1442, 1444, 1446, 1448. An air intake (not shown) is normally provided for an atomizer. In the example device 1400, each atomizer 1440, 1442, 1444, 1446, 1448 could have its own air intake or share a common air intake with other atomizers. Without any airflow control or vapor flow control, inhaling on the mouthpiece 1460 would draw air through not only any atomizers 1440, 1442, 1444, 1446, 1448 that are currently in use to produce vapor, but also through inactive atomizers, thereby diluting a stream of vapor with additional air. The valves 1450, 1452, 1454, 1456, 1458, and/or one or more gas regulators provided on an air intake side of one or more of the atomizers 1440, 1442, 1444, 1446, 1448 could be useful in avoiding or limiting such dilution. Such valves/regulators could be controlled to be open only if an associated atomizer 1440, 1442, 1444, 1446, 1448 is in use. For example, the controller 1430 could provide power to each atomizer 1440, 1442, 1444, 1446, 1448 and respective associated (normally closed) valves/regulators, so that an airflow path is open only when the associated atomizer is active.

According to some embodiments, one chamber (1410 as an example) could store a vaporization substance that includes an active ingredient or substance, and another chamber (1412 as an example) could store a vaporization substance that includes an antidote for the active ingredient or substance. Either or both of these vaporization substances could include a dry substance, a liquid, a gel and/or a wax. The active ingredient or substance could include a cannabinoid such as THC. The antidote could also include a cannabinoid such as CBD.

CBD is one example of an antidote to an active ingredient or substance in the form of THC. Other antidotes and active ingredients or substances are also possible. In general, an antidote as referenced herein is intended to encompass a substance that may reduce, reverse, or otherwise counteract one or more effects of an active ingredient or substance. An antidote could also or instead include, for example, a substance that could interfere with a cannabinoid receptor such as the CB1 receptors and/or CB2 receptors.

In the above example, the antidote stored in chamber 1412 allows a user to reduce or negate the effects of the vaporization substance stored in chamber 1410. For example, the user might feel that they have inhaled too much of an active ingredient or substance and/or otherwise be uncomfortable with its effects. The antidote could be administered by the device 1400 in any of a variety of ways. For example, the user input devices 1434 could include an input device, such as a button, switch or dial, to controllably administer the antidote. Controllably administering the antidote could include vaporizing the vaporization substance containing the antidote in the atomizer 1442, so that the user can inhale the produced vapor. The same input device 1434, and/or one or more other input devices, could provide dosage control by stopping, reducing, or otherwise limiting the inhalation of the active ingredient or substance of the vaporization substance that is stored in chamber 1410, and/or other chambers 1414, 1416, 1418.

One or more user input devices 1434 could be controllable to reduce a flow of vapor from the atomizer 1440, thereby reducing the flow of vapor containing the active ingredient or substance, and/or increase a flow of vapor from the atomizer 1442, thereby increasing the flow of vapor containing the antidote. This could be achieved, for example, by reducing or turning off power to the atomizer 1440, and/or increasing or turning on power to the atomizer 1442. One or more user input devices 1434 could also or instead adjust the valve 1420 to reduce or stop the movement of the vaporization substance including the active ingredient or substance to the atomizer 1440, and/or adjust the valve 1422 to increase the movement of the vaporization substance including the antidote to the atomizer 1442. Moreover, one or more user input devices 1434 could adjust the valve 1450 to reduce a flow of vapor including the active ingredient or substance from the atomizer 1440 to the mouthpiece 1460, and/or adjust the valve 1452 to increase a flow of vapor including the antidote from the atomizer 1442 to the mouthpiece.

FIG. 14 is a block diagram of an example vaporization device. An example multi-chamber, multi-atomizer cartridge, which could be used in an embodiment to implement multiple chambers in a vaporization device of the type shown in FIG. 14, is shown in FIGS. 15 to 19.

FIG. 15 is a plan view of a multi-chamber cartridge 1500, FIG. 16 is an isometric view of the multi-chamber cartridge 1500, FIG. 17 is an isometric view of a cap 1700 for the multi-chamber cartridge 1500, FIG. 18 is a bottom view of the cap 1700, and FIG. 19 is a cross-sectional view of the cap 1700, along line C-C in FIG. 18. Various features referenced in the description below are shown in one or more of these drawings.

The multi-chamber cartridge 1500 includes two chambers 1502, 1504. Two chambers of equal size are shown by way of example. There could be more than two chambers. Chambers could all be of the same size, or one or more chambers could have a different size from one or more other chambers. The chambers 1502, 1504 are positioned on a base 1506 and could be held in place by friction fit and/or some other type of releasable or fixed engagement. A partition wall 1508 is provided between the chambers 1502, 1504 to separate the respective vaporization substances stored in the chambers. The chambers 1502, 1504 also have respective outer walls 1503, 1505.

Examples of materials from which each chamber 1502, 1504 could be made are provided elsewhere herein. The chambers 1502, 1504 could include non-recloseable chambers, recloseable chambers, or both a non-recloseable chamber and a recloseable chamber. More generally, a multi-chamber cartridge or multi-chamber vaporization device could include one or more non-recloseable chambers and/or one or more recloseable chambers.

A cap, an example of which is shown in FIGS. 17 to 19, could engage the chambers 1502, 1504 and the stems 1516, 1526, and both seal the chambers 1502, 1504 and also hold them in place on the base 1506. The cap 1700 includes a channel 1702, through which vapor can be inhaled by a user.

The cap 1700 could be held onto the top (opposite the base 1506) of the multi-chamber cartridge 1500 through a snap, clip, or other type of releasable or fixed engagement. Alternatively, the connection between the cap 1700 and the multi-chamber cartridge 1500 could be non-releasable. A gasket or other sealing member could be provided between the top of the outer walls 1503, 1505, and a bottom of the cap 1700 to seal the vaporization substances in the multi-chamber cartridge 1500. A gasket or other sealing member could also or instead be provided between the partition 1508 and the bottom of the cap 1700 and/or between the stems 1516, 1526 to seal the vaporization substances in their respective chambers.

Stems 1516, 1526 are provided in the chambers 1502, 1504, respectively. The stems 1516, 1526 could be implemented as described elsewhere herein, with reference to FIGS. 1 and 2, for example. When the cap 1700 is engaged with or coupled to the multi-chamber cartridge 1500, the stems 1516, 1526 engage with respective channels 1704, 1706 in the cap. Although the cross-sections of the channels 1704, 1706 are illustrated as being cylindrical in FIGS. 18 and 19, the channels 1704, 1706 could instead have a frustoconical cross-section near the bottom of the cap 1700. This frustoconical cross-section could make it easier for a user to align the stems 1516, 1526 with the channels 1704, 1706, and also provide a friction fit between the stems and channels. Alternately or in addition, the distal end of the stems 1516, 1526 could be chamfered to aid in alignment and create a friction fit with the channels 1704, 1706. A gasket or other sealing member could be provided between the stems 1516, 1526 and the channels 1704, 1706.

The stem 1516 engages, and is in fluid communication with, a valve 1514 in the example shown. The valve 1514 also engages with, and is in fluid communication with, an atomizer 1510. The atomizer 1510 is engaged with the base 1506. Although any or all of these engagements could be releasable, the stem 1516, the valve 1514, the atomizer 1510 and the base 1506 could be permanently attached together. Sidewalls enclosing the valve 1514 and the atomizer 1510 could be formed with the stem 1516 as an integrated single physical component. The stem 1526, valve 1524, and atomizer 1520 have a similar structure. The stems 1516, 1526, the atomizers 1510, 1520, and the valves 1514, 1524 could be implemented as described elsewhere herein.

The base 1506 supplies power to the atomizers 1510, 1520. The base 1506 could be implemented in a similar manner to the base 106 (FIGS. 1 and 2) as described elsewhere herein. The base 1506 engages, and could also be coupled to, the chambers 1502, 1504 via an engagement. The engagement could be a fixed connection or a releasable engagement. In some embodiments, the base 1506 could be a form of a cover that releasably engages the chambers 1502, 1504 and seals one or both of the chambers. The chambers 1502, 1504 could have respective bottom walls, but in other embodiments the base 1506 seals the base end of one or more chambers.

The atomizers 1510, 1520 convert vaporization substances from the chambers 1502, 1504, respectively, into vapor. The atomizers 1510, 1520 could be implemented as described elsewhere herein, with reference to FIGS. 1 and 2, for example. The atomizers 1510, 1520 are inside the respective chambers 1502, 1504 in the example cartridge 1500, but in other embodiments one or more atomizers could be outside of but in fluid communication with a chamber.

Vaporization substances could be drawn into or otherwise provided to each atomizer 1510, 1520 through the intake holes 1512, 1522. One or more regulators could also be provided to regulate flows of one or more of the vaporization substances to the atomizers 1510, 1520. For example, valves could be implemented to control flow of vaporization substances from the chambers 1502, 1504 to the atomizers 1510, 1520. Such valves could be similar to the valves 1420, 1422, 1424, 1426, 1428 described above with reference to FIG. 14.

The valves 1514, 1524 are adjustable to control the flow of vapor from the atomizers 1510, 1520 to the stems 1516, 1526. The valves 1514, 1524 could be similar to the valves 1450, 1452, 1454, 1456, 1458 described above with reference to FIG. 14.

Vapor produced in the atomizers 1510, 1520 flows through the stems 1516, 1526 into the channels 1704, 1706, and is combined in the channel 1702 of the cap 1700. Alternatively, the cap 1700 might not include the channel 1702, and instead a user could inhale directly from two separate channels that extend through the cap, or through respective first and second mouthpieces.

Although not illustrated, the bottom walls of the chambers 1502, 1504 could include an engagement structure engage with a complementary engagement structure on the base 1506. Example engagement structures are described elsewhere herein, and could be implemented in multi-chamber, multi-atomizer cartridges or vaporization devices.

Engagement structures could restrict a cartridge or vaporization device to a particular model or type of chamber. This could be particularly useful in embodiments that provide an antidote chamber. For example, a particular engagement structure could limit a certain position or location on a base or in a vaporization device to a specific chamber or cartridge that stores an antidote. That position or location could then be dedicated to the antidote and uniquely associated with a particular user input device, such as a panic button. The panic button could be physically coupled to, hardwired to, or otherwise coupled to or associated with the antidote chamber position and/or to components such as a vaporization substance regulator, atomizer, and/or vapor regulator for that chamber position, to dispense the antidote. Such a panic button could also or instead reduce or shut down supply of any or all other vaporization substances and/or vapor produced from such substances. A panic button is shown by way of example at 1550.

Each chamber 1502, 1504 could include an indicator of its contents, and the base 1506 could be capable of detecting such indicators. Pins as described elsewhere herein represent an example of such indicators. Operating parameters such as voltage, current, and/or power level supplied to the atomizers 1510, 1520 (including duty cycle, for example), vaporization substance intake rates, and/or flow rates through one or move valves 1514, 1524, could be controlled based on the indicators. Other forms of indicator and detection could be provided in other embodiments.

As noted above, at least one chamber of a multi-chamber cartridge could store a vaporization substance that includes an antidote for an active ingredient or substance. In some implementations of the multi-chamber cartridge 1500, the chamber 1502 could store a vaporization substance including the active ingredient or substance, and the chamber 1504 could store a vaporization substance including the antidote. Indicators on the chambers 1502, 1504 could be used by the base 1506 to determine the type of vaporization substances stored in each chamber.

If a user of the multi-chamber cartridge 1500 wishes to reduce or negate the effects of the active ingredient or substance, then the user could use an input device to controllably administer antidote vapor and/or stop the flow of vapor containing the active ingredient or substance. This input device could be provided on the base 1506, as shown at 1550 for example, or elsewhere on a vaporization device. In an example of a vaporization substance that includes an active ingredient or substance in the chamber 1502 and an antidote in the chamber 1504, the input device could be controllable to reduce the flow of vapor from the atomizer 1510 in order to reduce the flow of vapor containing the active substance, and/or increase the flow of vapor from the atomizer 1520 in order to increase the flow of vapor containing the antidote. This could be achieved, for example, by reducing the flow of power to the atomizer 1510, and/or increasing the flow of power to the atomizer 1520. The input device could also or instead adjust the valve 1512 to reduce the movement of the vaporization substance including the active substance to the atomizer 1510, and/or adjust the valve 1522 to increase the movement of the vaporization substance including the antidote to the atomizer 1520. The input device could additionally or instead adjust the valve 1514 to reduce a flow of vapor including the active substance from the atomizer 1510 to the stem 1516, and/or adjust the valve 1524 to increase a flow of vapor including the antidote from the atomizer 1520 to the stem 1526.

FIGS. 15 to 19 represent one example embodiment. Other embodiments are contemplated. For example, a chamber need not necessarily conform tightly to other components. Tight conformance between components may be preferred to make the most efficient use of limited physical space, but in other embodiments multiple cylindrical chambers could be assembled to the same base, for example. In another embodiment, multiple cartridges, each with its own base and atomizer, could be installed in a vaporization device that has multiple ports or connectors to a battery and/or other components in a battery compartment.

The partition wall 1508 could be a single wall as shown, or could be provided in multiple sections, as described above with reference to FIG. 8, for example. Any of various other features disclosed herein, such as a movable partition wall or movable partition wall sections, one or more sealing members attached to or otherwise carried by a partition wall or partition wall sections, and/or structures such as grooves or channels as guides to placement of partition walls at certain positions, could also or instead be provided.

Chambers such as 1502, 1504 need not share a partition wall or partition wall sections, and could instead be self-contained, as also disclosed elsewhere herein.

Embodiments described above relate primarily to multi-chamber apparatus such as cartridges or vaporization devices. Other embodiments, including methods, are also contemplated.

FIG. 20, for example, is a flow diagram illustrating a method 2000 according to an embodiment. The example method 2000 involves an operation 2002 of providing chambers to store vaporization substances, an operation 2004 of providing a mixer to mix the vaporization substances, and an operation 2006 of providing one or more atomizers to atomize vaporization substances and/or a vaporization substance mixture. These operations 2002, 2004 and 2006 are shown separately for illustrative purposes, but need not be separate operations in all embodiments. For example, a vaporization device could include a mixer and an atomizer, and could also be sold with vaporization substance chambers as well. A vaporization device that is usable with multiple chambers, or components thereof, could potentially be provided separately from the chambers, which could be purchased separately, for example, and therefore the operation of providing chambers is optional in at least some embodiments.

The chambers, mixer, and/or atomizer(s) could be provided at 2002, 2004, 2006 by actually manufacturing these components. Any of these components, and/or other components, could instead be provided by purchasing or otherwise acquiring the components from one or more suppliers.

At least some components or parts thereof could be provided in different ways. Different cartridge parts, such as chambers, bases, covers, and atomizers, for example, could be provided by manufacturing one or more parts and purchasing one or more other parts, or by purchasing different parts from different suppliers.

A mixer that is provided at 2004 could include an active mixer to receive and actively mix vaporization substances to form a vaporization substance mixture, or a passive mixer to receive vaporization substances and mix the vaporization substance with multiple mixing elements and form a vaporization substance mixture. Either of these types of mixer could include a mixing channel to receive the vaporization substances.

An active mixer could be or include a stirring element, such as any one or more of: a stirring element to be driven by an electric motor, a magnetic stirring element, and an acoustic stirring element, as disclosed by way of example elsewhere herein. Examples of passive mixers disclosed herein include mixing elements including a splitter to split a received stream of the vaporization substances into multiple streams and a combiner coupled to the splitter to combine the streams, wells, ridges, grooves, linear elements, and helical elements.

In some embodiments, components such as the mixer provided at 2004 and the atomizer(s) provided at 2006, and possibly the chambers provided at 2002, are provided in the form of a pre-assembled vaporization device. In other embodiments, components are not necessarily assembled. FIG. 20 therefore also illustrates an operation 2008 of assembling components. This could involve, for example, arranging an atomizer in fluid communication with the mixer, such as by installing the atomizer and/or the mixer in a vaporization device or cartridge. Chambers could also or instead be assembled at 2008, by installing the chambers in a vaporization device or cartridge or otherwise arranging the chambers in fluid communication with the mixer in some embodiments.

The example method 2000 is illustrative of one embodiment. Examples of various ways to perform the illustrated operations, additional operations that may be performed in some embodiments, or operations that could be omitted in some embodiments, could be inferred or apparent from the description and drawings, for example. Further variations may be or become apparent.

For instance, other components could be provided and/or assembled. Examples of operations involving such other components include providing a channel for fluid communication with an atomizer, providing a mouthpiece for fluid communication with the channel, and providing the vaporization substances. A method could also or instead involve providing regulators to control movement of the vaporization substances to the mixer, and possibly also arranging the regulators in fluid communication with the mixer. A power controller to control power to the atomizer could also or instead be provided and coupled to the atomizer.

One or more components, such as chambers, could be refilled or replaced as shown at 2010.

Not all embodiments necessarily involve all of the operations shown in FIG. 20. For example, not all embodiments employ vaporization substance mixing. A method could involve providing a channel and providing multiple chambers at 2002 to store respective vaporization substances, with the chambers including a first chamber to store a first vaporization substance that includes an active ingredient or substance and a second chamber to store a second vaporization substance that includes an antidote for the active ingredient or substance. Assembly at 2008, if these components are not pre-assembled, could involve arranging the first chamber and the second chamber in fluid communication with the channel.

Providing the chambers at 2002 could involve providing at least one of the first chamber and the second chamber with an engagement structure to engage with a complementary engagement structure of the apparatus, in which case assembly at 2008 could involve arranging the at least one of the first chamber and the second chamber with the engagement structure engaging with the complementary engagement structure of the apparatus.

At 2006, a first atomizer to vaporize the first vaporization substance and/or a second atomizer to vaporize the second vaporization substance could be provided. The atomizer(s) could be installed at 2008, for example, by arranging the first atomizer in fluid communication with the first chamber and the channel, and/or arranging the second atomizer in fluid communication with the second chamber and the channel.

Providing the first atomizer at 2006 could involve providing the first atomizer inside the first chamber. Similarly, a method could also or instead involve providing the second atomizer at 2006 inside the second chamber. In such embodiments, 2002 and 2006 could be performed together, by providing one or more cartridges, for example.

In some embodiments, providing a channel involves providing multiple channels, including a first channel for fluid communication with the first atomizer and a second channel for fluid communication with the second atomizer. Providing multiple channels could involve providing a first mouthpiece including the first channel and/or providing a second mouthpiece including the second channel. In another embodiment, providing multiple channels involves providing a mouthpiece that includes a third channel for fluid communication with the first channel and the second channel.

A method could involve providing a first vaporization substance regulator to control movement of the first vaporization substance from the first chamber and/or providing a second vaporization substance regulator to control movement of the second vaporization substance from the second chamber. Installation of the regulator(s) at 2008 could involve arranging the first vaporization substance regulator in fluid communication with the first chamber and/or arranging the second vaporization substance regulator in fluid communication with the second chamber. At least one of the first vaporization substance regulator and the second vaporization substance regulator could include: a wick, a valve, a pump, and a mechanical feed structure.

Vapor regulators could also or instead be provided. For example, a method could include providing a first vapor regulator to control a flow of vapor, from vaporization of the first vaporization substance, in the channel; and/or providing a second vapor regulator to control a flow of vapor, from vaporization of the second vaporization substance, in the channel. Assembly at 2008 could then involve arranging the first vapor regulator in fluid communication with the channel; and/or arranging the second vapor regulator in fluid communication with the channel. At least one of the first vapor regulator and the second vapor regulator could be or include a valve or a pump.

Another component that could be provided and assembled or installed is a power controller. In some embodiments, a method involves providing a first power controller to control power to the first atomizer and/or providing a second power controller to control power to the second atomizer. A method could also involve coupling the first power controller to the first atomizer and/or coupling the second power controller to the second atomizer.

A method could involve providing an input device to controllably administer the antidote.

Other features disclosed herein could also apply to method embodiments. For example, the first vaporization substance and/or the second vaporization substance could be or include at least one of: a liquid, a gel and a wax. In some embodiments, the active substance is or includes a cannabinoid. The antidote could be or include cannabidiol.

Other variations of methods associated with manufacturing or otherwise producing a multi-chamber apparatus such as a cartridge or a vaporization device may be or become apparent.

User methods are also contemplated. FIG. 21 is a flow diagram illustrating a method according to another embodiment.

The example method 2100 involves an optional operation 2102 of installing or replacing one or more chambers. A user need not necessarily install or replace chambers every time a vaporization substance mixture is to be vaporized. The example method 2100 also involves an operation 2104 of initiating supply of one or more vaporization substances, to a mixer for example, and an operation 2106 of activating one or more atomizers. These operations could involve operating one or more input devices such as a control button or switch or even just inhaling on a mouthpiece. The operations at 2104 and 2106 are shown separately in FIG. 21 solely for illustrative purposes, and need not necessarily be separate operations.

Similarly, inhaling vapor is shown separately at 1508, but in some embodiments inhaling on a mouthpiece initiates vaporization substance flow, mixing, and vaporization.

The dashed arrows in FIG. 21 illustrate that multiple doses of a vaporization substance or mixture could be vaporized, and that available vaporization substances could be changed by installing or replacing one or more chambers.

The example method 2100, like the example method 2000, is an illustrative and non-limiting example. Various ways to perform the illustrated operations, additional operations that may be performed in some embodiments, or operations that could be omitted in some embodiments, could be inferred or apparent from the description and drawing or otherwise be or become apparent.

For example, vaporization of a first vaporization substance to produce vapor that includes an active ingredient or substance could be initiated through one or more actions at 2104, 2106, by operating a power button and/or inhaling on a vaporization device mouthpiece, for example. The vapor that includes the active ingredient or substance could be inhaled at 2108, and vaporization of a second vaporization substance that includes an antidote for the active ingredient or substance could subsequently be initiated in the same or a different way at 2104, 2106, by operating a panic button or other input device, for example. This produces vapor that includes the antidote, which can then be inhaled at 2108.

It should be appreciated that the drawings and description herein are intended solely for illustrative purposes, and that the present invention is in no way limited to the particular example embodiments explicitly shown in the drawings and described herein.

What has been described is merely illustrative of the application of principles of embodiments of the present disclosure. Other arrangements and methods can be implemented by those skilled in the art.

Features disclosed herein, whether in the context of apparatus, methods, and/or other embodiments, need not necessarily be implemented in combination with each other. In general, features may be implemented individually or in any of various combinations.

As an example, consider the user input device safety feature that is also referenced herein as a panic button feature, to enable a user to dispense or administer an antidote for reducing an effect of an active ingredient or substance that has been inhaled by the user. Such a feature may be significant and important in its own right, without necessarily being dependent upon or implemented in conjunction with at least some of the other features disclosed herein. In an apparatus embodiment, for example, a basic implementation of user input device-based antidote administration may include a channel, multiple chambers in fluid communication with the channel, and an input device. The chambers include a first chamber to store a first vaporization substance that includes an active ingredient or substance, and a second chamber to store a second vaporization substance that includes an antidote for the active ingredient or substance. The input device is operable by a user to controllably dispense or administer the antidote.

User safety may be of paramount importance, to aid in avoiding a prolonged effect of over-consumption of an active ingredient and/or in reducing recovery time from active ingredient consumption, for example. Ease of use and reliability may be important factors in respect of input device-based antidote delivery. A user's mental and/or motor skills may be impaired if the user has over-consumed an active ingredient, and accordingly an input device that is easy to use to effectively administer an antidote may be especially preferred.

Some examples of input devices and how such devices may be used to administer antidote are provided elsewhere herein. For example, an input device could be in the form of a button, switch, or dial. Buttons are shown at 144 in FIGS. 1-2 and at 1550 in FIGS. 15-16. The examples provided above for those buttons, and/or other examples of input devices provided herein, could be used to implement an input device for administering an antidote. Although the button 144 may be used to control operation of a vaporization device as described by way of example above, in some embodiments device operation is automatic and responsive to a user inhaling on a mouthpiece, and the button 144 is an antidote or panic button that is easily operable by a user.

It is possible for an input device to have multiple purposes or functions.

Considering again the button 144 as an example, the button could be pressed and held to turn a vaporization device on or off, and “clicked” (pushed but not held) to administer antidote. A dedicated input device that is used only to control administration of an antidote may be preferred, however, so that a user need not recall which of different operating methods must be applied to administer the antidote.

Buttons, switches, and dials are not the only forms of input devices that may be implemented. Other examples include pressure sensors, which may include but not be limited to various types of buttons, rotatable structures that may include but not be limited to various types of dials, and elements such as accelerometers and strain gauges to detect movement or manipulation of vaporization devices or parts thereof in a certain way. Any input device that is capable of receiving user input may be used as an input device to control administration of an antidote.

An input device may be carried by the first chamber, the second chamber, a base that is coupled to the first chamber and/or to the second chamber, a battery housing, or a mouthpiece that is coupled to the first chamber and/or to the first chamber, for example. A rotatable structure such as a dial or rotatable ring may be located on a vaporization device battery housing, between the battery housing and a cartridge base, on the base, between a base and the chamber(s) to which the base is coupled, on a chamber, between a chamber and a mouthpiece, or on a mouthpiece.

A battery housing example is shown in FIGS. 1-2 at 144, and a base example is shown in FIGS. 15-16 at 1550. In FIGS. 15-16, the input device 1550 is carried by a base 1506 that is coupled to two chambers 1502, 1504, but in other embodiments a separate base may be coupled to an antidote chamber and an input device is carried by the antidote chamber base.

Another embodiment includes a pressure sensor, which could dispense the antidote and/or adjust the supply of active vaporization substances once detected pressure exceeds a particular threshold value. A pressure sensor could be embedded within or otherwise carried by a mouthpiece, for example, and the sensor threshold value could be set to detect when a user bites on the mouthpiece. This is an example of an input device that is not only easy for a user to use, but is also unlikely to be inadvertently triggered by a user during normal operation of a vaporization device.

A pressure sensor could be embedded within or otherwise carried by outer walls of one of a chamber, a base, or another part of a vaporization device such that squeezing the chamber or base activates antidote administration.

An accelerometer on or in a vaporization device may detect a user input in the form of slamming the vaporization device on a surface to activate antidote administration.

A user input device may include one or more strain gauges to detect a user bending, pulling, or pushing on a device as user input to administer antidote.

Another potential aspect of ease of use relates to identification of an input device and/or mode of operation to administer an antidote. A panic button or other input device may be identified with one or more special markings or indicia, such as “SOS” or a cross icon, on the input device itself and/or a vaporization device for example, to indicate an antidote or emergency function. A panic button or other input device for administering antidote may be illuminated in a special color or otherwise indicated. One or more markings or indicia, on an input device and/or a vaporization device for example, may also or instead provide antidote or emergency operating instructions.

Examples of how an antidote may be controllably administered are also provided at least above, and any of those examples may be implemented in user input device-based administration of an antidote.

Operation of an input device may have any of various effects. For example, in an embodiment operation of an input enables one of: control to increase a flow of vapor from vaporization of the a vaporization substance that includes an antidote; and control to increase power to an atomizer of the vaporization substance, to controllably administer the antidote. Other examples are also provided herein, such as reducing flow of another vaporization substance or vapor from vaporization of another vaporization substance.

Regarding reliability of input device-based antidote administration, an input device that is uniquely associated with an antidote chamber may be preferred. A unique association may reduce the likelihood of the input device being inadvertently used to deliver anything other than antidote. For example, an input device could be physically coupled, hardwired, and/or otherwise coupled to any one or more of various parts of a vaporization device, such as a chamber in which a vaporization substance that includes the antidote is stored, a vaporization substance regulator in fluid communication with the second chamber to control flow of the vaporization substance from the chamber, an atomizer associated with the chamber to vaporize the vaporization substance, a vapor regulator to control a flow of vapor from vaporization of the vaporization substance, a power source, and a controller.

Engagement structures are used in some embodiments to provide or maintain a unique association between an input device and an antidote. As described herein, engagement structures may restrict a cartridge or vaporization device to a particular model or type of chamber. For example, a particular engagement structure could limit a certain position or location on a base or in a vaporization device to a specific chamber or cartridge that stores a vaporization substance that includes an antidote, and an input device may be uniquely coupled to or otherwise uniquely associated with that position, location, and/or chamber. The antidote position, location, and/or chamber may thus be specific and dedicated to the antidote and uniquely associated with a particular input device.

In general, one-to-one specificity between an antidote and a single input device may be preferred. This may be useful, for example, in minimizing the number of operating instructions or modes that a user would need to recall in an emergency or other situation in which an antidote is to be administered. In other embodiments, several input devices, including different types of input devices for example, are provided in order to give a user multiple options for administering an antidote. Therefore, one or more input devices for controllably administering an antidote may be provided.

Method embodiments may also or instead be subject to such variations, in which some but not necessarily all disclosed features are implemented. For example, a method may involve providing a channel, providing a plurality of chambers in fluid communication with the channel, and providing an input device. In an embodiment, the chambers include a first chamber to store a first vaporization substance and a second chamber to store a second vaporization substance, the second vaporization substance includes an antidote for an active ingredient or substance in the first vaporization substance, and the input device is provided to controllably administer the antidote.

Any of various other features disclosed herein may be implemented in conjunction with such a method. For example, the input device is uniquely associated with the second chamber in some embodiments. The second chamber may include an engagement structure to engage with a complementary engagement structure, of the apparatus, that is incompatible with the first chamber. Such an arrangement of engagement structures may aid in providing or maintaining a unique association between an input device and an antidote chamber.

The input device may also or instead be coupled to one of: the second chamber, a vaporization substance regulator in fluid communication with the second chamber to control flow of the second vaporization substance from the second chamber, an atomizer associated with the second chamber to vaporize the second vaporization substance, a vapor regulator to control a flow of vapor from vaporization of the second vaporization substance, a power source, and a controller. In some embodiments the input device is carried by the first chamber, the second chamber, a base that is coupled to the first chamber, a base that is coupled to the second chamber, a mouthpiece that is coupled to the first chamber, or a mouthpiece that is coupled to the first chamber.

Examples of such an input device include at least a button, a switch, a slider, a dial, a rotatable structure, a pressure sensor, an accelerometer, and a strain gauge, and other types of input devices may also or instead be provided. Operation of an input device may enable such features as: control to increase a flow of vapor from vaporization of the second vaporization substance; and control to increase power to an atomizer of the second vaporization substance, to controllably administer the antidote. Other features may also or instead be provided or enabled by operation of an input device, and further examples are provided elsewhere herein.

While the present invention has been described with reference to specific features and embodiments thereof, various modifications and combinations can be made thereto without departing from the invention. The description and drawings are, accordingly, to be regarded simply as an illustration of some embodiments of the invention as defined by the appended claims, and are contemplated to cover any and all modifications, variations, combinations or equivalents that fall within the scope of the present invention. Therefore, although the present invention and potential advantages have been described in detail, various changes, substitutions and alterations can be made herein without departing from the invention as defined by the appended claims. Moreover, the scope of the present application is not intended to be limited to the particular embodiments of any process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. An apparatus comprising:
a channel;
a plurality of chambers in fluid communication with the channel, the plurality of chambers comprising:
a first chamber to store a first vaporization substance, the first vaporization substance comprising an active substance; and
a second chamber to store a second vaporization substance, the second vaporization substance comprising an antidote for the active substance.

2. The apparatus of claim 1, wherein the first vaporization substance comprises at least one of: a dry substance, a liquid, a gel and a wax, wherein the second vaporization substance comprises at least one of: a dry substance, a liquid, a gel and a wax.

3. (canceled)

4. The apparatus of claim 1, wherein the active substance comprises a cannabinoid, wherein the antidote comprises cannabidiol.

5. (canceled)

6. The apparatus of claim 1, wherein at least one of the first chamber and the second chamber comprises an engagement structure to engage with a complementary engagement structure of the apparatus.

7. The apparatus of claim 1, further comprising:
a first atomizer, in fluid communication with the first chamber and the channel, to vaporize the first vaporization substance; and
a second atomizer, in fluid communication with the second chamber and the channel, to vaporize the second vaporization substance.

8-9. (canceled)

10. The apparatus of claim 7, wherein the channel comprises a plurality of channels, the first atomizer being in fluid communication with a first channel of the plurality of channels and the second atomizer being in fluid communication with a second channel of the plurality of channels, wherein the first channel comprises a first mouthpiece and the second channel comprises a second mouthpiece.

11. (canceled)

12. The apparatus of claim 7, wherein the channel comprises a plurality of channels, the first atomizer being in fluid communication with a first channel of the plurality of channels and the second atomizer being in fluid communication with a second channel of the plurality of channels, wherein the first channel and the second channel are in fluid communication with a third channel of the plurality of channels, the third channel comprising a mouthpiece.

13. The apparatus of claim 1, further comprising:
a first vaporization substance regulator to control movement of the first vaporization substance from the first chamber; and
a second vaporization substance regulator to control movement of the second vaporization substance from the second chamber, wherein the first vaporization substance regulator and the second vaporization substance regulator provide dosage control for the apparatus.

14-19. (canceled)

20. The apparatus of claim 1, further comprising:
a first vapor regulator to control a flow of vapor, from vaporization of the first vaporization substance, in the channel; and
a second vapor regulator to control a flow of vapor, from vaporization of the second vaporization substance, in the channel, wherein the first vapor regulator and the second vapor regulator provide dosage control for the apparatus.

21-27. (canceled)

28. The apparatus of claim 1, further comprising an input device to controllably administer the antidote, wherein the input device is uniquely associated with the second chamber.

29. The apparatus of claim 28, wherein the input device comprises a button, a switch, a slider, a dial, a rotatable structure, a pressure sensor, an accelerometer, or a strain gauge.

30-37. (canceled)

38. A method comprising:
providing a channel;
providing a plurality of chambers to store respective vaporization substances, the plurality of chambers comprising:
a first chamber to store a first vaporization substance, the first vaporization substance comprising an active substance; and
a second chamber to store a second vaporization substance, the second vaporization substance comprising an antidote for the active substance.

39. The method of claim 38, further comprising:
arranging the first chamber and the second chamber in fluid communication with the channel.

40. The method of claim 38, wherein the first vaporization substance comprises at least one of: a dry substance, a liquid, a gel and a wax, wherein the second vaporization substance comprises at least one of: a dry substance, a liquid, a gel and a wax.

41. (canceled)

42. The method of claim 38, wherein the active substance comprises a cannabinoid, wherein the antidote comprises cannabidiol.

43. (canceled)

44. The method of claim 38, wherein providing the plurality of chambers comprises providing at least one of the first chamber and the second chamber with an engagement structure to engage with a complementary engagement structure of a vaporization device.

45. (canceled)

46. The method of claim 38, further comprising:
providing a first atomizer to vaporize the first vaporization substance; and
providing a second atomizer to vaporize the second vaporization substance.

47-49. (canceled)

50. The method of claim 46, wherein providing the channel comprises providing a plurality of channels, the plurality of channels comprising a first channel for fluid communication with the first atomizer and a second channel for fluid communication with the second atomizer, wherein providing a plurality of channels comprises providing a first mouthpiece comprising the first channel and providing a second mouthpiece comprising the second channel.

51. (canceled)

52. The method of claim 46, wherein providing the channel comprises providing a plurality of channels, the plurality of channels comprising a first channel for fluid communication with the first atomizer and a second channel for fluid communication with the second atomizer, wherein providing a plurality of channels comprises providing a mouthpiece comprising a third channel for fluid communication with the first channel and the second channel.

53. The method of claim 38, further comprising:
providing a first vaporization substance regulator to control movement of the first vaporization substance from the first chamber; and
providing a second vaporization substance regulator to control movement of the second vaporization substance from the second chamber.

54-55. (canceled)

56. The method of claim 38, further comprising:
providing a first vapor regulator to control a flow of vapor, from vaporization of the first vaporization substance, in the channel; and
providing a second vapor regulator to control a flow of vapor, from vaporization of the second vaporization substance, in the channel.

57-60. (canceled)

61. The method of claim 38, further comprising:
providing an input device to controllably administer the antidote.

62. A method of use of the apparatus of claim 1, the method comprising:
initiating vaporization of the first vaporization substance to produce vapor comprising the active substance;
inhaling, through the channel, the vapor comprising the active substance;
initiating vaporization of the second vaporization substance to produce vapor comprising the antidote;
inhaling, through the channel, the vapor comprising the antidote.

63. A method comprising:
initiating vaporization of a first vaporization substance that comprises an active substance, to produce vapor comprising the active substance;
inhaling the vapor comprising the active substance;
initiating vaporization of a second vaporization substance that comprises an antidote for the active substance, to produce vapor comprising the antidote;
inhaling the vapor comprising the antidote.

64. The method of claim 63, wherein initiating vaporization of the second vaporization substance comprises operating an input device to controllably administer the antidote.
